(19) United States
(12) Patent Application Publication
(10) Pub. No.: US 2009/0112024 A1
(43) Pub. Date: Apr. 30, 2009

(54) STABILIZATION OF HYDROXYLAMINE CONTAINING SOLUTIONS AND METHOD FOR THEIR PREPARATION

(76) Inventor: Wai Mun Lee, Fremont, CA (US)

Correspondence Address: MORGAN LEWIS & BOCKIUS LLP, 1111 PENNSYLVANIA AVENUE NW, WASHINGTON, DC, 20004, US

(21) Appl. No.: 12/260,474

(22) Filed: Oct. 29, 2008

Related U.S. Application Data
(60) Provisional application No. 61/000,727, filed on Oct. 29, 2007.

(51) Int. Cl. C07C 239/08

(52) U.S. Cl. 564/301

(57) ABSTRACT

The invention relates to the use of amidoximes for prevention of or stabilization of hydroxylamine compounds against undesired decomposition.

### FIELD OF THE INVENTION

The present invention relates to stabilized compositions containing hydroxylamine and methods of their preparation. More specifically, the present invention relates to the use of amidoximes for the stabilization of hydroxylamine compounds against undesired decomposition.

### BACKGROUND

Aqueous hydroxylamine is widely used in chemical syntheses, but its instability greatly limits its utility in situations where storage is necessary and in reactions where product purity is important. The problem of instability of aqueous solutions containing hydroxylamine is particularly serious when these solutions are obtained by ion-exchange techniques.

Just in the last decade, catastrophic explosions resulting in the loss of lives occurred in two instances during manufacturing processes involving hydroxylamine free base due to the presence of unstabilized hydroxylamine. See FIG. 1.

Since the introduction of hydroxylamine into a semiconductor cleaning process by Lee (see e.g., U.S. Pat. No. 5,279,771 and U.S. Pat. No. 5,334,332), the use of hydroxylamine free base is now extending to chemical mechanical planarization in semiconductor processes.

U.S. Pat. Nos. 7,172,744; 7,105,078; 7,045,655; 7,029,557; 6,942,762; 6,908,956; 6,867,327; 6,758,990; 6,534,681; 6,524,545; 6,153,799; 5,906,805; 5,872,295; 5,837,107; 5,808,150; 5,783,161; 4,778,669; 4,645,579; 4,634,584; 4,629,613; 4,601,800; 4,576,804; 4,551,318 and others have described manufacturing and stabilization processes for the production of hydroxylamine free base which were developed by or acquired by BASF Aktiengesellschaft in Germany since the mid 1980's.

WO 2005016817 describes manufacturing processes for the production of hydroxylamine free base developed by Showa Denko K.K in Japan. Other references describe lists of stabilizers used during hydroxylamine free base manufacturing processes. The stabilizers may be known stabilizers such as those disclosed on pages 19-21 of WO 2005016817, and include the following: 8-hydroxyquinoline; N-hydroxyethylethylenediamine-N,N,N′-triacetic acid; glycine; ethylenediaminetetraacetic acid; cis-1,2-diaminocyclohexane-N,N,N′,N′-tetraacetic acid; trans-1,2-diaminocyclohexane-N,N,N′,N′-tetraacetic acid; N,N′-di(2-hydroxybenzyl)ethylenediamine-N,N′-diacetic acid; N-hydroxyethyliminodiacetic acid; N,N′-dihydroxyethylglycine; diethylenetriaminepentaacetic acid; ethylenebis(oxyethylenenitrilo)tetraacetic acid; bishexamethylenetriaminepentaacetic acid; hexamethylenediaminetetraacetic acid; triethylenetetraminehexaacetic acid; tris(2-aminoethyl)aminehexaacetic acid; iminodiacetic acid; polyethyleneimine; polypropyleneimine; o-aminoquinoline; 1,10-phenanthroline; 5-methyl-1,10-phenanthroline; 5-chloro-1,10-phenanthroline; 5-phenyl-1,10-phenanthroline; hydroxyanthraquinone; 8-hydroxyquinoline-5-sulfonic acid; 8-hydroxymethylquinoline; thioglycolic acid; thiopropionic acid; 1-amino-2-mercapto-propionic acid; 2,2-dipyridyl; 4,4-dimethyl-2,2-dipyridyl; ammoniumthiosulfate; benzotriazole; flavone; morin; quercetin; gossypetin; robinetin; luteolin; fisetin; apigenin; galangin; chrysin; flavonol; pyrogallol; oxyanthraquinone; 1,2-dioxyanthraquinone; 1,4-dioxyanthraquinone; 1,2,4-trioxyanthraquinone; 1,5-dioxyanthraquinone; 1,8-dioxyanthraquinone; 2,3-dioxyanthraquinone; 1,2,6-trioxyanthraquinone; 1,2,7-trioxyanthraquinone; 1,2,5,8-tetraoxyanthraquinone; 1,2,4,5-S-pentaoxyanthraquinone; 1,6-S-dioxy-3-methyl-6-methoxyanthraquinone; quinalizarin; flavan; 2,3-dihydrohexono-1,4-lactone; 8-hydroxyquinaldine; 6-methyl-5-hydroxyquinaldine; 5,8-dihydroxyquinaldine; anthocyan; pelargonidin; cyanidin; delphinidin; paeonidin; petunidin; malvidin; catechin; sodium thiosulfate; nitrilotriacetic acid; 2-hydroxyethyldisulfide; 1,4-dimercapto-2,3-butanediol; thiamine hydrochloride; catechol; 4-tert-butylcatechol; 2,3-dihydroxynaphthalene; 2,3-dihydroxybenzoic acid; 2-hydroxypyridine-N-oxide; 1,2-dimethyl-3-hydroxypyridin-4-one; 4-methylpyridine-N-oxide; 6-methylpyridine-N-oxide; 1-methyl-3-hydroxypyridin-2-one; 2-mercaptobenzothiazole; 2-mercaptocyclohexylthiazole; 2-mercapto-6-tertbutylcyclohexylthiazole; 2-mercapto-4,S-dimethylthiazoline; 2-mercaptothiazoline; 2-mercapto-5-tert-butylthiazoline; tetramethylthiuramdisulfide; tetra-n-butylthiuramdisulfide; N,N′-diethylthiuramdisulfide; tetraphenylthiuramdisulfide; thiuramdisulfide; thiourea; N,N′-diphenylthiourea; di-o-tolylthiourea; ethylenethiourea; thiocetamide; 2-thiouracil; thiocyanuric acid; thioformamide; thioacetamide; thiopropionamide; thiobenzamide; thionicotinamide; thioacetanilide; thiobenzanilide; 1,3-dimethylthiourea; 1,3-diethyl-2-thiourea; 1-phenyl-2-thiourea; 1,3-diphenyl-2-thiourea; thiocarbazide; thiosemicarbazide; 4,4-dimethyl-3-thiosemicarbazide; 2-mercaptoimidazoline; 2-thiohydantoin; 3-thiourazole; 2-thiouramil; 4-thiouramil; thiopentanol; 2-thiobarbituric acid; thiocyanuric acid; 2-mercaptoquinoline; 2-mercapto-4H-3,1-benzoxazine; 2-mercapto-4H-3,1-benzothiazine; thiosaccharin; 2-mercaptobenzimidazole; trimethylphosphite; triethylphosphite; triphenylphosphite; trimethylphosphine; triethylphosphine; and triphenylphosphine.

Cis-1,2 diaminocyclohexane-N,N,N′,N′-tetraacetic acid is a commonly used stabilizer in commercially available hydroxylamine free base solutions.

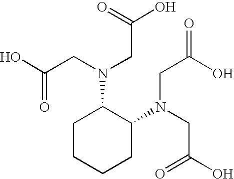

Even with the required amount of stabilizer present in the 50% hydroxylamine free base, the desired effect of preventing decomposition of the hydroxylamine due to metal impurities may not be achieved. When a higher concentration of the stabilizer is utilized, removal of the excess stabilizer may be required.

A commercially available sample of hydroxylamine free base (50%) solution was obtained from BASF to demonstrate the ease of decomposition of hydroxylamine as currently stabilized. The study demonstrated that hydroxylamine can be easily decomposed when contaminated with trace metal ions, such as iron (III) in the form of ferric chloride. The experimental procedure used in this demonstration is described herein.

Commercial utilization of hydroxylamine free base (50%) solution introduces metal impurities to the solution. This will, in turn, accelerate the decomposition of hydroxylamine free base in such systems, particularly when a cleaning solution containing hydroxylamine free base is used in semiconductor manufacturing processes, despite the extremely low levels of trace metals in the hydroxylamine free base (50%) solution. The trace metals specifications for the hydroxylamine free base (50%) solution are typically less than 10 ppb. FIG. 2 (a reproduction of FIG. 9 of U.S. Pat. No. 5,334,332 to Lee) shows the percent hydroxylamine activity of various compositions. The compositions for L, N and R are as follows: (see col. 12, lines 25-49 of U.S. Pat. No. 5,334,332):

| | | 2-(2-amino- | | 1,2 dihydroxy- |
|---|---|---|---|---|
| Cleaning | Hydroxylamine | ethoxy) | | benzene |
| Composition | (neat) Wt % | ethanol | Water | (catechol) |
| L | 25% | 50% | 25% | 0% |
| N | 20% | 55% | 20% | 5% |
| R | 15% | 70% | 15% | 0% |

The solutions are kept at room temperature for 80 days. Composition N, the most stable composition of the group, contains the chelating agent, catechol, which acts as an additional stabilizer in the hydroxylamine solution. This confirms that trace metals have been introduced into the composition through mixing with other compounds which could potentially contain high levels of metal impurities. In this case, the chemical compound which introduced the metals impurities is an alkanolamine. See FIG. 2.

The use of catechol in such formulated products is followed by ACT and TOK in their product formulations.

An effective stabilizer for hydroxylamine-containing solutions should be at least substantially soluble in aqueous solutions. The majority of substrates being used in the semiconductor cleaning process, including, for example, in post-CMP cleaning, contain metallic-etch residue removal, Such metallic contamination could accelerate the decomposition of hydroxylamine-containing solutions. Proper complexing agents, sometimes called chelating agents, are required to stabilize the degradation of hydroxylamine. Much is known about metal-chelating functionality in which a central metal ion to be attached by coordination links to two or more nonmetal atoms (ligands) in the same molecule. Heterocyclic rings are formed with the central metal atom as part of each ring. When the complex becomes more soluble in the solution, it functions in the cleaning process. If the complexed product is not soluble in the solution, it becomes a passivating agent by forming an insoluble film on top of the metal surface. The complexing agents currently in use, such as, glycolic acid, glyoxylic acid, lactic acid, and phosphonic acid, are acidic and have a tendency to attack the metals and metal oxides, such as copper and copper oxide, thus undermining their efficacy.

This situation presents a problem for formulators who aim to produce a stable hydroxylamine containing cleaning solution, which has selectivity only to a metal oxide and not to the metal itself, e.g., in an application involving a metal, such as copper. Accordingly, there is a need for complexing agents that are not aggressive toward metal substrates, and yet effectively chelate metal ion residues created during semiconductor manufacturing processes. Such chelating agents can also function as stabilizers for hydroxylamine-containing compositions.

The present invention addresses these problems.

### SUMMARY OF THE INVENTION

In one aspect, the present invention is directed to an aqueous solution comprising hydroxylamine and an amidoxime compound, wherein the amidoxime compound is present in an amount effective to prevent degradation or stabilize the hydroxylamine. In an exemplary embodiment, the amidoxime compound is prepared from the reaction between hydroxylamine and a nitrile compound.

In an exemplary embodiment, the nitrile compounds are derived from the cyanoethylation of nucleophilic compounds with acrylonitrile or another unsaturated nitrile. The nucleophilic compounds may be selected from the group consisting of

(a) compounds containing one or more —OH or —SH groups, such as water, alcohols, phenols, oximes, hydrogen sulphide and thiols;

(b) compounds containing one or more —NH— groups, for example, ammonia, primary and secondary amines, hydrazines, and amides;

(c) ketones or aldehydes possessing a —CH—, —CH₂—, or —CH₃ group adjacent to the carbonyl group; and

(d) compounds such as malonic esters, malonamide and cyanoacetamide, in which a —CH or —CH₂— group is situated between —CO₂R, —CN, or —CONH— groups.

One embodiment of the invention is a method of preventing degradation of and/or stabilizing hydroxylamine, comprising contacting the hydroxylamine with an effective amount of an amidoxime compound, wherein the amidoxime compound is prepared from a reaction of hydroxylamine and a nitrile compound. The hydroxylamine may be present as an aqueous solution. In one embodiment, the amidoxime has any one of the following structures:

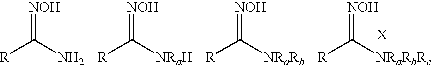

or tautomers thereof, wherein X is a counterion and R, Rₐ, R_{b} and R_{c} are independently selected from alkyl, heteroalkyl, aryl and heteroaryl, wherein the alkyl, heteroalkyl, aryl and heteroaryl are optionally substituted. R may be optionally a substituted alkyl group or a substituted heteroalkyl group. In one embodiment, R has more than 10 carbons. In another embodiment of the invention, the amidoxime has a molecular weight of above 200.

In another embodiment of the invention, the amidoxime has the following structure:

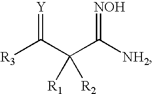

wherein R₁ and R₂ are independently selected from hydrogen, alkyl, heteroalkyl, aryl and heteroaryl; R₃ is alkyl, heteroalkyl, aryl and heteroaryl, wherein the alkyl, heteroalkyl, aryl and heteroaryl are optionally substituted; and Y is O, NH or NOH.

In yet another embodiment, the amidoxime has the following structure:

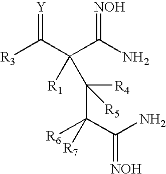

wherein R₁, R₂, R₄, R₅, R₆ and R₇ are independently selected from hydrogen, alkyl, heteroalkyl, aryl and heteroaryl; R₃ is alkyl, heteroalkyl, aryl and heteroaryl, wherein the alkyl, heteroalkyl, aryl and heteroaryl are optionally substituted; and Y is O, NH or NOH.

In other embodiments, the amidoxime may be selected from the group consisting of 1,2,3,4,5,6-hexakis-O-[3-(hydroxyamino)-3-iminopropyl hexitol; 3,3′,3″,3′″-(ethane-1,2-diylbis(azanetriyl))tetrakis(N′-hydroxypropanimidamide); 3,3′-(ethane-1,2-diylbis(oxy))bis(N′-hydroxypropanimidamide); 3-(diethylamino)-N′hydroxypropanimidamide; 3,3′-(piperazine-1,4-diyl)bis(N′-hydroxypropanimidamide); 3-(2-ethoxyethoxy)-N′-hydroxypropanimidamide; 3-(2-(2-(dimethylamino)ethoxy)ethoxy)-N′-hydroxypropanimidamide; N′-hydroxy-3-(phenylamino)propanimidamide; 3,3′,3″-nitrilotris(N′-hydroxypropanimidamide); 3,3′-(2,2-bis((3-(hydroxyamino)-3-iminopropoxy)methyl)propane-1,3-diyl)bis(oxy)bis(N-hydroxypropanimidamide); 3,3′-(2,2′-(methylazanediyl)bis(ethane-2,1-diyl)bis(oxy))bis(N′-hydroxypropanimidamide); N,N-bis(3-amino-3-(hydroxyimino)propyl)acetamide; 3,3′-(2-(N′hydroxycarbamimidoyl)phenylazanediyl)bis(N′-hydroxypropanimidamide); 3,3′-(2,2′-(3-amino-3-(hydroxyimino)propylazanediyl)bis(ethane-2,1-diyl))bis(oxy)bis(N′-hydroxypropanimidamide); N′,3-dihydroxypropanimidamide; NN′-hydroxyacetimidamide; N′-hydroxy-3-(methylamino)propanimidamide; 3,3′-azanediylbis(N′-hydroxypropanimidamide); 3-amino-3-(hydroxyimino)propanoic acid; 3-amino-3-(hydroxyimino)propanamide; N′1,N′10-dihydroxydecanebis(imidamide); N′-hydroxyisonicotinimidamide; 2-dihydroxyacetimidamide; 2-chloro-N′-hydroxyacetimidamide; 2-amino-N′-hydroxybenzimidamide; 2,2′-azanediylbis(N′-hydroxyacetimidamide); N′-hydroxy-1-oxo-1,3-dihydroisobenzofuran-5-carboximidamide; 3-aminoisoquinolin-1(4H)-one oxime; 3-(hydroxyamino)-3,4-dihydroisoquinolin-1-amine; N′-hydroxycinnamimidamide; 4-chloro-N′-hydroxybenzimidamide; and salts thereof.

In another embodiment of the invention, the nitrile compound is prepared from cyanoethylation of nucleophilic compounds with acrylonitrile. The nucleophilic compound may be selected from (1) compounds containing one or more —OH or —SH groups; (2) compounds containing one or more —NH— groups, (3) ketones or aldehydes possessing a —CH—, —CH₂—, or —CH₃ group adjacent to the carbonyl group; and (3) malonic esters, malonamide and cyanoacetamide. The compounds containing one or more —OH or —SH groups include but are not limited to e.g. alcohols, phenols, oximes, hydrogen sulphide and thiols. The compounds containing one or more —NH— groups include but are not limited to ammonia, primary and secondary amines, hydrazines, and amides.

An aspect of the invention is also directed to a method of stabilizing a solution comprising a hydroxylamine, the method including the step of adding to the solution at least one nitrile compound derived from the cyanoethylation of nucleophilic compounds with acrylonitrile.

It is to be understood that the foregoing general description and the following detailed description are exemplary and explanatory and are intended to provide further explanation of the invention as claimed.

### BRIEF DESCRIPTION OF THE FIGURES

The accompanying figures, which are included to provide a further understanding of the invention and are incorporated in and constitute part of this specification illustrate embodiments of the invention and together with the description serve to explain the principles of the invention.

FIG. 1 is a process flow sheet for the hydroxylamine free base manufacturing facility at Nissin Chemical Co. in Japan.

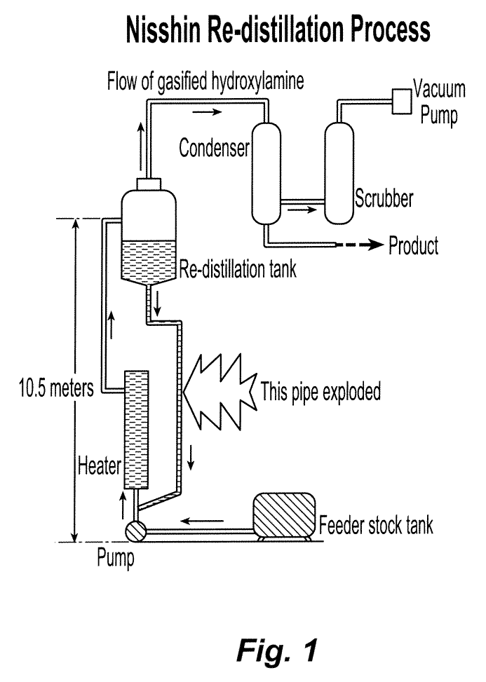

FIG. 2 is a reproduction of FIG. 9 of U.S. Pat. No. 5,334,332.

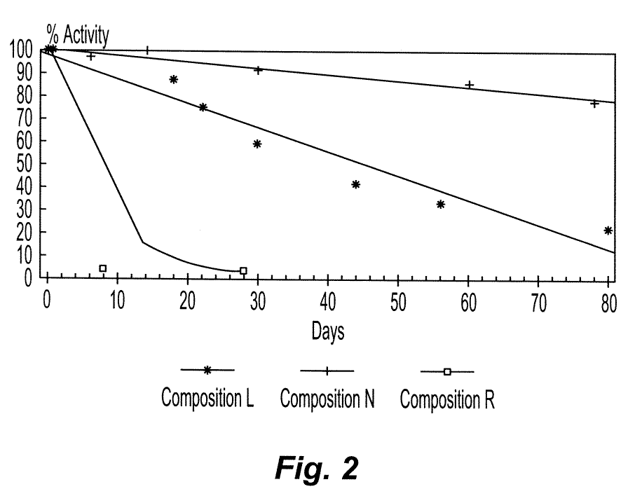

### DETAILED DESCRIPTION

One embodiment of the present invention is an aqueous composition comprising hydroxylamine and an amidoxime compound (i.e., a compound containing one or more amidoxime functional groups) wherein the amidoxime compound complexes with a metal (or a metal oxide) to prevent degradation of and/or stabilize the hydroxylamine.

In a particular embodiment, the hydroxylamine is in free base form. In another particular embodiment, the free base form is a 50% solution in water.

Optionally, the hydroxylamine containing composition further contains one or more organic solvents.

In one embodiment, the amidoxime compound stabilizes the hydroxylamine by preventing or diminishing the rate of decomposition of the hydroxylamine.

Optionally, the composition contains one or more surfactants.

Optionally, the composition contains one or more additional compounds containing functional groups which complex or chelate with metals or metal oxides.

Optionally, the composition contains one or more acids or bases.

In an exemplary embodiment, the composition contains from about 0.1% to about 99.99% of hydroxylamine as a free base 50% solution and from about 0.01% to about 99.9% of one or more amidoxime compounds (i.e., compounds with one or more amidoxime functional groups).

In an exemplary embodiment, the amidoxime compound may be used in combination with other chelating compounds or with compounds possessing other functional groups that provide a complexing or chelating function, such as hydroxamic acid, thiohydroxamic acid, N-hydroxyurea, N-hydroxycarbamate and/or N-nitroso-alkyl-hydroxylamine groups.

A greater number of amidoxime functional groups in a single molecule may be advantageous because it allows for multi-dentate binding. Multi-dentate binding is advantageous for a number of reasons—for example, because multi-dentate ligands tend to have higher association constants than mono-dentate ligands. A higher association constant is useful in, for example, facilitating the removal of hard-to-remove residues from the surface.

In some embodiments, the use of mono-dentate ligands is preferred in semiconductor processing, for example, for ease of their synthesis.

In other embodiments, water and/or solvent soluble ligands are preferred.

The amidoxime functional group has the following chemical formula:

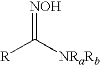

or salts thereof.

In a particular embodiment, Rₐ and R_{b} are independently hydrogen, alkyl, heteroalkyl, alkyl-aryl, or alkyl-heteroaryl groups. R is independently selected from alkyl, alkyl-aryl, or alkyl-heteroaryl groups. In these embodiments, chelation of the amidoxime to metal centers may be favored because, in reaction with a metal center, a proton can be lost from NRₐR_{b} so as to form a nominally covalent bond with the metal center.

In another embodiment, NRₐR_{b} is further substituted with R_{c}, to form a salt with the following chemical formula:

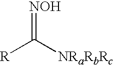

Any counter-ion may be used. Examples include, but are not limited to, chloride (Cl⁻), bromide (Br⁻), iodide (I⁻), sulfate (SO₄²⁻), PF₆⁻ or ClO₄⁻. R_{c}, may be hydrogen, alkyl, alkyl-aryl, or alkyl-heteroaryl groups.

Rₐ, R_{b} and/or R_{c} may optionally join together so as to form one or more heterocyclic rings.

The amidoxime compounds of the invention may exist as their tautomers as shown below in an exemplary embodiment:

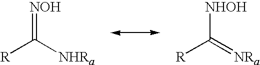

Compounds that exist mainly or wholly in this tautomeric (or resonance) form are included within the scope of the present invention.

In exemplary embodiments, the amidoxime compounds as described herein include the following compounds and their tautomers:

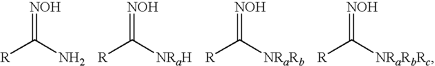

wherein R is as defined above and may optionally be connected to one or more of Rₐ, R_{b} and R_{c} to form a ring or rings.

In an exemplary embodiment, the amidoxime compound has the structure below in the form of a salt, wherein Alk is an alkyl group as defined below.

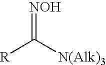

In this embodiment, the three alkyl groups are independently selected. In a particular embodiment, the alkyl group is methyl or ethyl.

In exemplary embodiments, R is defined as above. The alkyl group may be straight-chained or branched and may include unsaturated bonds (e.g., alkene and/or alkyne) in the chain.

The alkyl group may contain any number of carbon and hydrogen atoms and may be optionally be substituted with, but not limited to, alkyl, halo, aryl, heteroaryl, —OH, ═O, —NH₂, ═NH, —NHOH, ═NOH, —OPO(OH)₂, —SH, ═S or —SO₂OH. While alkyl groups having a lesser number of carbon atoms tend to be more soluble in polar solvents such as DMSO and water, alkyl groups having a greater number of carbons can have other advantageous properties, for example surfactant properties. In an exemplary embodiment, the alkyl group contains 1 to 10 carbon atoms, for example 1 to 6 carbon atoms. In another exemplary embodiment, the alkyl group contains 10 or more carbon atoms, for example 12 to 24 carbon atoms.

Examples of alkyl groups include, but are not limited to, methyl, ethyl, n-propyl, sec-propyl, cyclopropyl, n-butyl, sec-butyl, tert-butyl, cyclobutyl, pentyl (branched or unbranched), cyclopentyl, hexyl (branched or unbranched), cyclohexyl, heptyl (branched or unbranched), cycloheptyl, octyl (branched or unbranched), cyclooctyl, nonyl (branched or unbranched), and decyl (branched or unbranched).

Examples of amidoxime compounds containing alkyl groups include, but are not limited to:

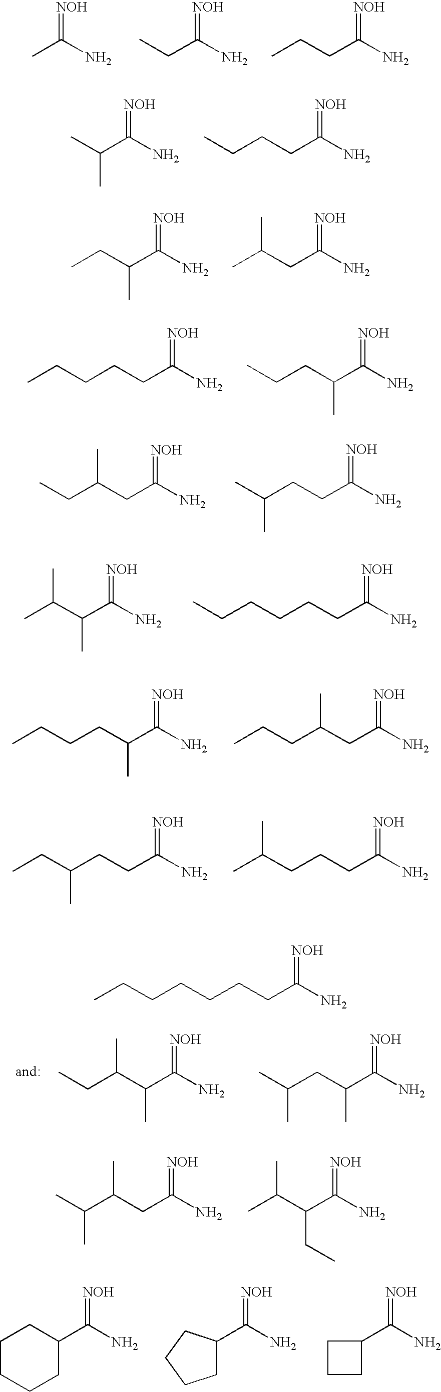

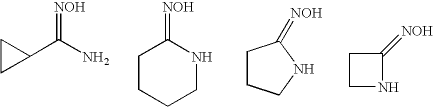

Examples further include alkylene, alkenyl or alkynyl linkers (R) appending two or more amidoxime compounds. In an exemplary embodiment, the di-amidoxime compound is:

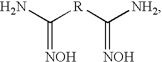

where R is an alkylene group. Examples of suitable groups include methylene, ethylene, propylene, butylene, etc. As defined herein the term “alkyl” is considered to encompass alkylene, alkenylene and alkynylene groups.

Specific examples of di-amidoxime compounds include, but are not limited to,

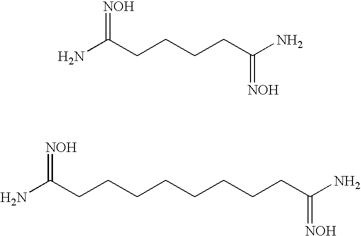

### Preparation of Di-Amidoxime Compounds is Provided in the Examples.

A specific example of an alkyne-containing amidoxime compound is as shown:

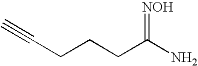

If the alkyl group is substituted with ═O, the alkyl group may comprise an aldehyde, a ketone, a carboxylic acid or an amide. In a particular embodiment, there is an enolizable hydrogen adjacent to the ═O, ═NH or ═NOH (i.e., there is a hydrogen in the alpha position to the carbonyl). The alkyl group may comprise the following functionality: —(CZ₁)-CH—(CZ₂)-, wherein Z₁ and Z₂ are independently selected from O, NH and NOH. The CH in this group is further substituted with hydrogen or an alkyl group or joined to the amidoxime functional group.

Thus, an alkyl group appending an amidoxime group may simply be substituted with, for example one or more independently-selected halogens, for example fluorine, chlorine, bromine or iodine. In one embodiment, the halogens are substituted at the antipodal (i.e., opposite) end of the alkyl group to the amidoxime group. This may, for example, provide surfactant activity, in particular, for example, if the halogen is fluorine. A specific example of an amidoxime group substituted with a halogen-substituted alkyl group is as shown:

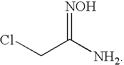

Compounds that are substituted in a β position are conveniently synthesized from readily-available starting materials.

Examples of such compounds include, but are not limited to

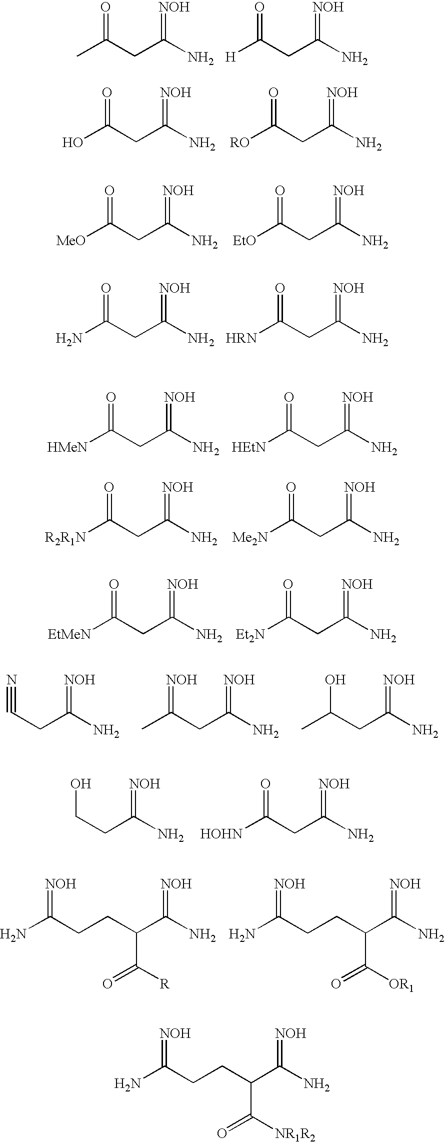

wherein R₁ and R₂ are independently-selected alkyl, aryl or heteroaryl groups or hydrogen atoms.

Specific examples of substituted alkyl amidoxime molecules include, but are not limited to:

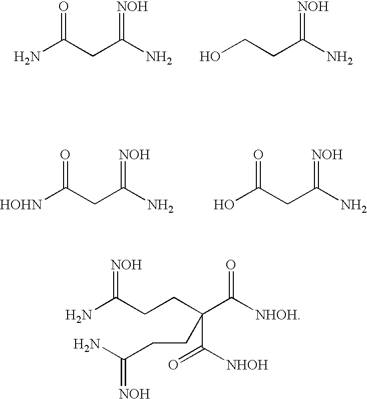

It should be noted that some of these molecules can exist as different isomers. For example:

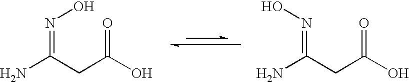

The different isomers can be differentiated by carbon-13 NMR.

In an exemplary embodiment, the amidoxime has the following structure:

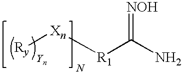

where “n” varies from 1 to N and y varies from 1 to Yₙ; N varies from 0 to 3; Yₙ varies from 0 to 5. In this formula, R₁ is an alkylene group; R_{y} is independently selected from alkyl, or heteroalkyl, alkyl-aryl and alkyl-heteroaryl groups, or adjoins R₁ so to form a heterocycle with the directly appending Xₙ. R₁ may also be a direct bond, so that the amidoxime group is connected directly to the one or more heteroatoms. Xₙ is a heteroatom or a group of heteroatoms selected from boron, nitrogen, oxygen, silicon, phosphorus and sulphur. Each heteroatom or group of heteroatoms and each alkyl group is independently selected from one another. The above formula includes an amidoxime group directly bearing an alkyl group. The alkyl group is substituted with N independently-selected heteroatoms or groups of heteroatoms. Each heteroatom or group of heteroatoms is itself substituted with one or more independently-selected alkyl groups or heteroalkyl groups.

In an exemplary embodiment, X is oxygen. In this case, X may be part of an ether group (—O—), an ester (—O—CO—), —O—CO—O—, —O—CO—NH—, —O—CO—NR₂—, —O—CNH—, O—CNH—O—, —O—CNH—NH—, —O—CNH—NR₂—, —O—CNOH—, —O—CNOH—O—, —O—CNOH—NH— or —O—CNOH—NR₂—, wherein R₂ is independently selected from an alkyl group, heteroalkyl group, aryl group, alkyl-aryl group, heteroaryl group and alkyl-heteroaryl group.

In another exemplary embodiment, X is a nitrogen atom. In this case, X may be part of one of the following groups: —NR₂H, —NR₂—, —NR₂R₃— (with an appropriate counter-ion), —NHNH—, —NH—CO—, —NR₂—CO—, —NH—CO—O—, —NH—CO—NH—, —NH—CO—NR₂—, —NR₂—CO—NH—, —NR₂—CO—NR₃—, —NH—CNH—, —NR₂—CNH—, —NH—CNH—O—, —NH—CNH—NH—, —NH—CNH—NR₂—, —NR₂—CNH—NH—, —NR₂—CNH—NR₃—, —NH—CNOH—, —NR₂—CNOH—, —NH—CNOH—O—, —NH—CNOH—NH—, —NH—CNOH—NR₂—, —NR₂—CNOH—NH—, —NR₂—CNOH—NR₃—. R₂ to R₃ are independently selected alkyl groups, heteroalkyl groups, or heteroaryl groups, wherein the heteroalkyl group and heteroaryl group may be unsubstituted or substituted with one or more heteroatoms or group of heteroatoms or itself be substituted with another heteroalkyl group. If more than one hetero-substituent is present, the substituents are independently selected from one another unless they form a part of a particular functional group (e.g., an amide group).

In another exemplary embodiment, X is boron.

In another exemplary embodiment, X is phosphorus. In a particular embodiment, X is an —OPO(OH)(OR₂) group or an —OPO(OR₂)(OR₃) group.

In another exemplary embodiment, X is sulphur. In a particular embodiment, X is a sulfoxide or a sulfone.

Particular examples of heteroalkyl groups include, but are not limited to, azetidines, oxetane, thietane, dithietane, dihydrofuran, tetrahydrofuran, dihydrothiophene, tetrahydrothiophene, piperidine, pyrroline, pyrrolidine, tetrahydropyran, dihydropyran, thiane, piperazine, oxazine, dithiane, dioxane and morpholine. These cyclic groups may be directly joined to the amidoxime group or may be joined to the amidoxime group through an alkyl group.

The heteroalkyl group may be unsubstituted or substituted with one or more heteroatoms or group of heteroatoms or itself be substituted with another heteroalkyl group. If more than one hetero-substituent is present, the substituents are independently selected from one another unless they form a part of a particular functional group (e.g., an amide group). One or more of the substituents may be a halogen atom, including fluorine, chlorine, bromine or iodine, —OH, ═O, —NH₂, ═NH, —NHOH, ═NOH, —OPO(OH)₂, —SH, ═S or —SO₂OH. In one embodiment, the substituent is an oxime group (═NOH). The heteroalkyl group may also be itself substituted with one or more amidoxime functional groups.

If the heteroalkyl group is substituted with ═O, the heteroalkyl group may comprise an aldehyde, a ketone, a carboxylic acid or an amide. Preferably, there is an enolizable hydrogen adjacent to the ═O, ═NH or ═NOH (i.e., there is a hydrogen in the alpha position to the carbonyl). The heteroalkyl group may comprise the following functionality: —(CZ₁)-CH—(CZ₂)-, wherein Z₁ and Z₂ are independently selected from O, NH and NOH. The CH in this group is further substituted with hydrogen or an alkyl group or heteroalkyl group or joined to the amidoxime functional group.

Amines are versatile functional groups for use in the present invention, in part because of their ease of preparation. For example, by using acrylonitrile as described later, a variety of functionalized amines can be synthesized.

Particular embodiments include, but are not limited to:

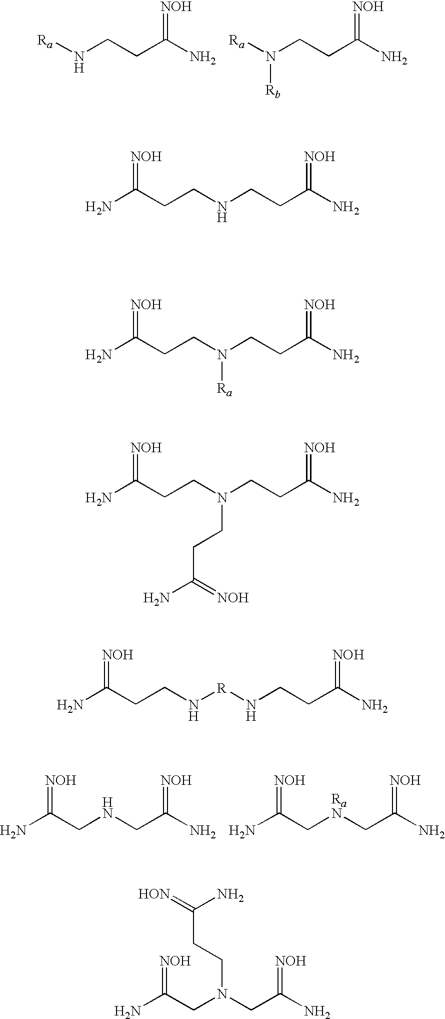

where Rₐ, and R_{b} are independently-selected hydrogen, alkyl, heteroalkyl, aryl, heteroaryl, alkyl-aryl, or alkyl-heteroaryl groups.

R may itself be an alkylene group or a heteroatom or group of heteroatoms. The heteroatoms may be unsubstituted or substituted with one or more alkyl groups.

R may be an aryl group. The term “aryl” refers to a group comprising an aromatic cycle. A particular example of an aryl substituent is a phenyl group.

The aryl group may be unsubstituted. A specific example of an amidoxime bearing an unsubstituted aryl is:

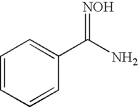

The aryl group may also be substituted with one or more alkyl groups, heteroalkyl groups or heteroatom substituents. If more than one substituent is present, the substituents are independently selected from one another.

Specific examples of amidoximes comprising a heteroalkyl group include:

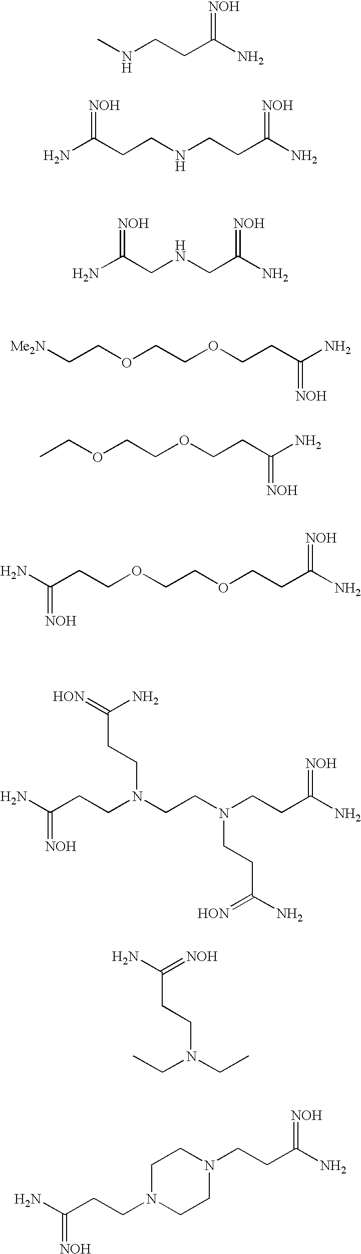

One or more of the heteroatom substituents may be for example, a halogen atom, including fluorine, chlorine, bromine or iodine, —OH, ═O, —NH₂, ═NH, —NHOH, ═NOH, —OPO(OH)₂, —SH, —S or —SO₂OH. In a particular embodiment, the substituent is an oxime group (═NOH).

Specific examples of substituted aryl amidoxime molecules include:

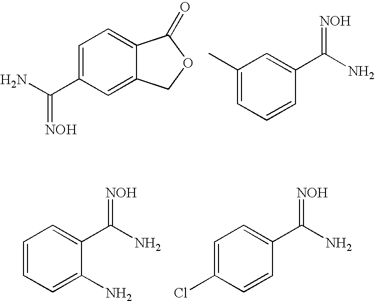

R may also be heteroaryl. The term heteroaryl refers to an aryl group containing one or more heteroatoms in its aromatic cycle. The one or more heteroatoms are independently-selected from, for example, boron, nitrogen, oxygen, silicon, phosphorus and sulfur. Examples of heteroaryl groups include, but are not limited to, pyrrole, furan, thiophene, pyridine, melamine, pyran, thiine, diazine and thiazine.

The heteroaryl group may be unsubstituted. A specific example of an unsubstituted heteroaryl amidoxime compound is:

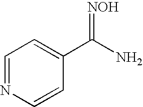

It should be noted that the heteroaryl group may be attached to the amidoxime group through its heteroatom, for example (the following molecule being accompanied by a counter anion):

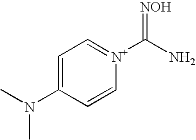

The heteroaryl group may be substituted with one or more alkyl groups, heteroalkyl groups or hetero-atom substituents. If more than one substituent is present, the substituents are independently selected from one another.

One or more of the hetero-atom substituents may be, for example, a halogen atom, including fluorine, chlorine, bromine or iodine, —OH, ═O, —NH₂, ═NH, —NHOH, ═NOH, —OPO(OH)₂, —SH, ═S or —SO₂OH. The one or more alkyl groups are the alkyl groups defined previously and the one or more heteroalkyl groups are the heteroalkyl groups defined previously.

Within the scope of the term aryl are alkyl-aryl groups. The term “alkyl-aryl” refers to an amidoxime group bearing (i.e., directly joined to) an alkyl (i.e., an alkylene group). The alkyl group is then itself substituted with an aryl group. Correspondingly, within the scope of the term heteroaryl are alkyl-heteroaryl groups.

Specific examples of unsubstituted alkyl-aryl amidoxime compounds are:

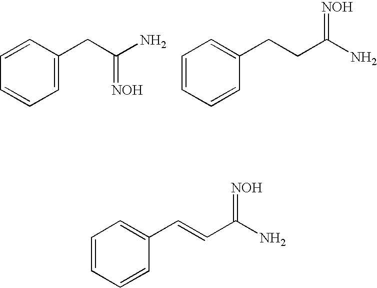

Alternatively, one or both of the alkyl group and the aryl/heteroalkyl group may be substituted. If the alkyl group is substituted, it may be substituted with one or more heteroatoms or groups containing heteroatoms. If the aryl/heteroalkyl group is substituted, it may be substituted with one or more alkyl groups, heteroalkyl groups or hetero-atom substituents. If more than one substituent is present, the substituents are independently selected from one another.

One or more of the heteroatom substituents may be, for example, a halogen atom, including fluorine, chlorine, bromine or iodine, —OH, ═O, —NH₂, ═NH, —NHOH, ═NOH, —OPO(OH)₂, —SH, ═S or —SO₂OH. In one embodiment, the substituent is an oxime group (═NOH). The alkyl group may also be itself substituted with one or more amidoxime functional groups.

If the alkyl group is substituted with ═O, the alkyl group may comprise an aldehyde, a ketone, a carboxylic acid or an amide. Preferably, there is an enolizable hydrogen adjacent to the ═O, ═NH or ═NOH (i.e. there is a hydrogen in the alpha position to the carbonyl). The alkyl group may comprise the following functionality: —(CZ₁)-CH—(CZ₂)-, wherein Z₁ and Z₂ are independently selected from O, NH and NOH. The CH in this group is further substituted with hydrogen or an alkyl group or heteroalkyl group or joined to the amidoxime functional group.

Within the scope of the term aryl are also heteroalkyl-aryl groups. The term “heteroalkyl-aryl” refers to an amidoxime group bearing (i.e. directly joined to) an heteroalkyl group. The heteroalkyl group is then itself substituted with an aryl group. Correspondingly, within the scope of the term heteroaryl are also heteroalkyl-aryl groups

The heteroalkyl group may be any alkyl group previously defined. The aryl/heteroaryl group may also be any aryl group previously defined.

Both the heteroalkyl group and the aryl/heteroaryl group may be unsubstituted. Alternatively, one or both of the heteroalkyl group and the aryl/heteroaryl group may be substituted. If the heteroalkyl group is substituted, it may be substituted with one or more heteroatoms or groups containing heteroatoms. If the aryl/heteroaryl group is substituted, it may be substituted with one or more alkyl groups, heteroalkyl groups or heteroatom substituents. If more than one substituent is present, the substituents are independently selected from one another.

One or more of the heteroatom substituents may be, for example, a halogen atom, including fluorine, chlorine, bromine or iodine, —OH, ═O, —NH₂, ═NH, —NHOH, ═NOH, —OPO(OH)₂, —SH, ═S or —SO₂OH. In an exemplary embodiment, the substituent is an oxime group (═NOH). The alkyl group may also be itself substituted with one or more amidoxime functional groups.

If the heteroalkyl group is substituted with ═O, the heteroalkyl group may comprise an aldehyde, a ketone, a carboxylic acid or an amide. Preferably, there is an enolizable hydrogen adjacent to the ═O, ═NH or ═NOH (i.e. there is a hydrogen in the alpha position to the carbonyl). The heteroalkyl group may comprise the following functionality: —(CZ₁)-CH—(CZ₂)-, wherein Z₁ and Z₂ are independently selected from O, NH and NOH. The CH in this group is further substituted with hydrogen or an alkyl group or heteroalkyl group or joined to the amidoxime functional group.

A preferred substituent to any type of R group is a tetra-valent nitrogen. In other words, any of the above groups may be substituted with —NRₐR_{b}R_{c} where Rₐ to R_{c}, are independently-selected R groups as defined herein. In one embodiment, Rₐ to R_{c}, are unsubstituted saturated alkyl groups having 1 to 6 carbon atoms. For example, one or more of (for example all of) Rₐ to R_{c} are methyl and/or ethyl. With this substituent, the tetra-valent nitrogen is preferably substituted in an antipodal position to the amidoxime group. For example, if R is a straight-chained unsubstituted saturated alkyl group of the form (CH₂)ₙ, then the tetra-valent nitrogen is at one end of the alkyl group and the amidoxime group is at the other end. In this embodiment, n is preferably 1, 2, 3, 4, 5 or 6.

In an exemplary embodiment, the present invention provides an amidoxime molecule that contains only one amidoxime functional group. In another embodiment, the present invention provides an amidoxime molecule containing two or more amidoxime functional groups. In fact, a large number of functional groups can be contained in a single molecule, for example if a polymer has repeating units having appending amidoxime functional groups. Examples of amidoxime compounds that contain more than one amidoxime functional groups have been described previously throughout the specification.

Amidoximes may be conveniently prepared from nitrile-containing molecules as follows:

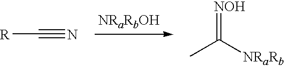

Typically, to prepare a molecule having Rₐ═R_{b}═H, hydroxylamine is used. If one or both of Rₐ and R_{b} in the desired amidoxime is not hydrogen, the amidoxime can be prepared either using the corresponding hydroxylamine or by further reacting the amidoxime once it has been formed. This may, for example, occur by intra-molecular reaction of the amidoxime.

Accordingly, amidoxime molecules containing more than one amidoxime functional groups can be conveniently prepared from precursors having more than one nitrile group. Specific amidoxime molecules having two amidoxime functional groups which have been synthesized in this way include:

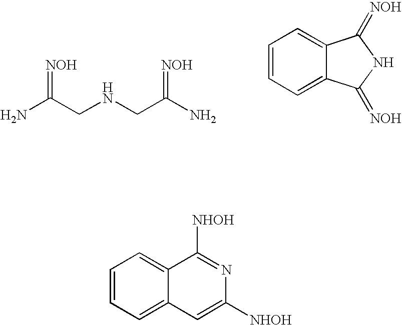

One exemplary method of forming the nitrile precursors to the amidoximes of the present invention is by nucleophilic substitution of a leaving group with a nucleophile. Nucleophiles are well known to the person skilled in the art, see for example the Guidebook to Mechanism in Organic Chemistry by Peter Sykes. Examples of suitable nucleophiles are molecules having an —OH, —SH, —NH or a suitable CH— group, for example one having a low pKₐ (for example, below about 15). For OH, SH and NH—, the hydrogen is optionally removed before acting as a nucleophile in order to augment its nucleophilicity. For CH—, the hydrogen is usually removed with a suitable base so that the resulting anion can act as a nucleophile.

Leaving groups are well known to the person skilled in the art. See, for example, the Guidebook to Mechanism in Organic Chemistry by Peter Sykes. Examples of suitable leaving groups include halogen (e.g., Cl, Br, I), O-tosyl, O-mesylate and other leaving groups well known to the person skilled in the art. Their ability to act as a leaving group may be enhanced by adding an acid, either protic or Lewis.

In one embodiment, a nitrile can be formed accordingly:

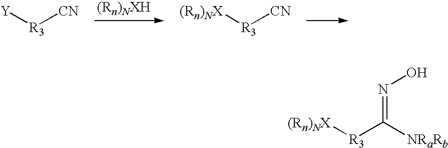

In this example, R₃ is independently selected from alkylene, heteroalkylene, arylene, heteroarylene, alkylene-heteroaryl, or alkylene-aryl groups. Rₙ is independently selected from hydrogen, alkyl, heteroalkyl, aryl, heteroaryl, alkyl-heteroaryl, or alkyl-aryl group. X may be any a nucleophile selected from O, S, N, and suitable C. N varies from 1 to 3. Y is a leaving group.

For XH═OH, the OH may be an alcohol group or may, for example, be part of a hemiacetal or carboxylic acid group.

For X═NH—, the NH may be part of a primary or secondary amine (i.e. NH₂ or NHR₅), NH—CO—, NH—CNH—, NH—CHOH— or —NHNR₅R₆ (wherein R₅ and R₆ are independently-selected alkyl, heteroalkyl, aryl, heteroaryl or alkyl-aryl).

For XH═CH—, wherein a stabilized anion may be formed. XH may be selected from but not limited to —CHCO—R₅, —CHCOOH, —CHCN, —CHCO—OR₅, —CHCO—NR₅R₆, —CHCNH—R₅, —CHCNH—OR₅, —CHCNH—NR₅R₆, —CHCNOH—R₅, —CHCNOH—OR₅ and —CHCNOH—NR₅R₆.

In a particular example, the nucleophile is:

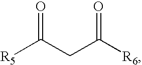

for example

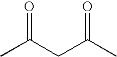

wherein R₅ and R₆ are independently-selected alkyl, heteroalkyl, aryl, heteroaryl or alkyl-aryl or a heteroatom optionally substituted with any of these groups. In an exemplary embodiment, either one or both of R₅ and R₆ are oxygen or nitrogen atoms optionally independently substituted with alkyl, heteroalkyl, aryl, heteroaryl, alkyl-heteroaryl or alkyl-aryl groups, for example:

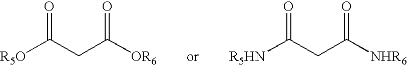

The compounds may also be formed by any type of nucleophilic reaction using any of the above nucleophiles.

In an exemplary embodiment, the following reaction is used for producing nitrile precursors for amidoxime compounds:

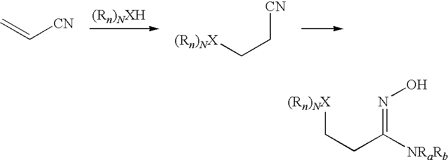

In this example, X bears N independently-selected substituents, wherein N is defined as above. Each Rₙ is independently chosen from hydrogen, alkyl, heteroalkyl, aryl, heteroaryl and alkylaryl as previously defined. X is a nucleophile. The acrylonitrile may be substituted as desired.

For example, the acrylonitrile may have the following formula:

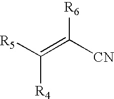

wherein R₄, R₅ and R₆ are independently selected from hydrogen, heteroatoms, heterogroups, alkyl, heteroalkyl, aryl, alkyl-aryl, alkyl-heteroaryl and heteroaryl.

The present invention also relates to amidoxime compounds for use in semiconductor processing, optionally prepared by the addition of a nucleophile to an unsubstituted or substituted acrylonitrile. Once nucleophilic addition to the acrylonitrile has occurred, the intermediate can be functionalized using standard chemistry known to the person skilled in the art:

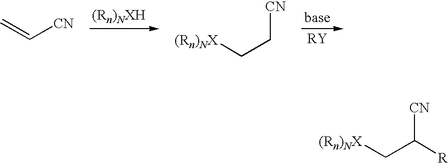

where Y is a leaving group.

Examples of simple nucleophiles with show the adaptability of this reaction include:

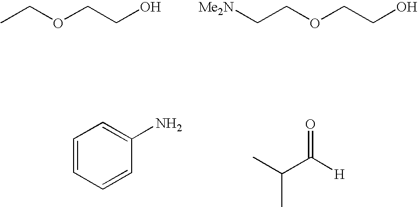

This reaction is particularly versatile, especially when applied to the synthesis of multidentate amidoxime compounds; (i.e., molecules containing two or more amidoxime functional groups). For example, it can be used to functionalize compounds having two or more NH groups. In one particular example, the reaction can be used to functionalize a molecule containing two or more primary amines:

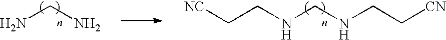

where n is 1 or more, for example 1 to 24.

Further functionalization of a primary amine is possible. For example, a tetradentate amidoxime, for example the functional equivalent of EDTA, may be conveniently formed:

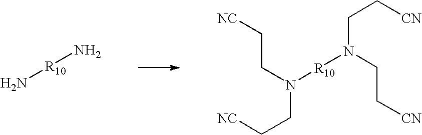

wherein R₁₀ is alkylene, heteroalkylene, arylene or heteroarylene. In an alternative embodiment, R₁₀ is a direct bond—i.e., the starting material is a hydrazine. An example of this reaction where R₁₀ is —CH₂CH₂— is provided in the examples.

In another exemplary embodiment, a molecule having two or more secondary amines can be functionalized:

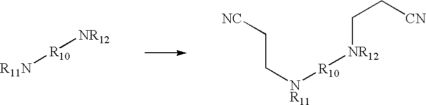

where R₁₀ is defined as above and R₁₁ and R₁₂ are independently selected alkyl, heteroalkyl, aryl or heteroaryl. An embodiment where R₁₀ is a direct bond is also contemplated.

For example, the secondary amines can be part of a cyclic system:

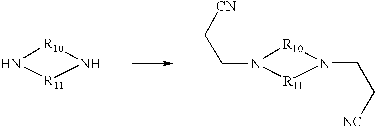

where R₁₀ and R₁₁ are defined above. In an exemplary embodiment, a common solvent used in semiconductor processing can be functionalized with amidoxime functional groups. For example:

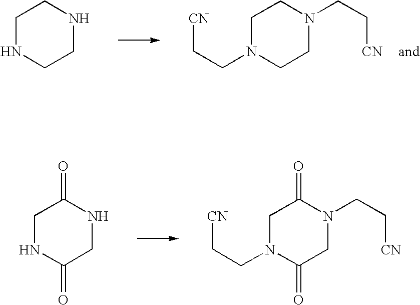

Details of these reactions are contained in the examples.

Similarly, an oxygen nucleophile may be used to provide nitrile precursors to amidoxime molecules. In one embodiment, the nucleophile is an alcohol:

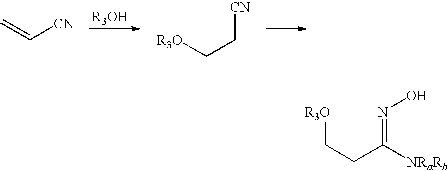

where R₃ is alkyl, heteroalkyl, aryl or heteroaryl.

The polyalcohol compounds may be functionalized. Poly-alcohols are molecules that contain more than one alcohol functional group. As an example, the following is a polyalcohol:

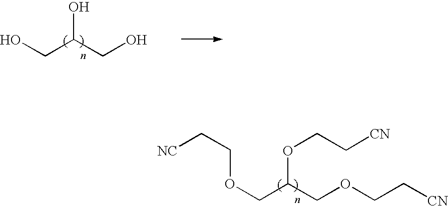

wherein n is 0 or more, for example 0 to 24. In one example, n is 0 (glycol). In another example, n is 6 (sorbitol).

In another example, the polyalcohol forms part of a polymer. For example, reaction may be carried out with a polymer comprising polyethylene oxide. For example, the polymer may contain just ethylene oxide units, or may comprise polyethylene oxide units as a copolymer (i.e. with one or more other monomer units). For example, the polymer may be a block copolymer comprising polyethylene oxide. For copolymers, especially block copolymers, the polymer may comprise a monomer unit not containing alcohol units. For example, the polymer may comprise blocks of polyethylene glycol (PEG). Copolymer (e.g., block copolymers) of polyethylene oxide and polyethylene glycol may be advantageous because the surfactant properties of the blocks of polyethylene glycol can be used and controlled.

Carbon nucleophiles can also be used. Many carbon nucleophiles are known in the art. For example, an enol group can act as a nucleophile. Harder carbon-based nucleophiles can be generated by deprotonation of a carbon. While many carbons bearing a proton can be deprotonated if a strong enough base is provided, it is often more convenient to be able to use a weak base to generate a carbon nucleophile, for example NaOEt or LDA. As a result, in one embodiment, a CH group having a pKₐ of 20 or less, for example 15 or less, is deprotonated to form the carbon-based nucleophile.

An example of a suitable carbon-based nucleophile is a molecule having the beta-diketone functionality (it being understood that the term beta-diketone also covers aldehydes, esters, amides and other C═O containing functional groups. Furthermore, one or both of the C═O groups may be replaced by NH or NOH).

For example:

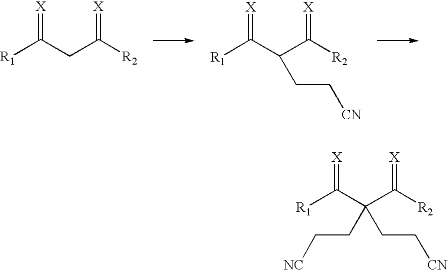

where R₁ and R₂ are independently selected alkyl groups, heteroalkyl groups, aryl groups, heteroaryl groups and heteroatoms.

A specific example of this reaction sequence where R₁═R₂═OEt is given in the examples. Nitrile groups act to lower the pKₐ of hydrogens in the alpha position. This in fact means that sometimes control of reaction conditions is preferably used to prevent a cyano compound, once formed by reaction of a nucleophile with acrylonitrile, from deprotonating at its alpha position and reacting with a second acrylonitrile group. For example, selection of base and reaction conditions (e.g., temperature) can be used to prevent this secondary reaction. However, this observation can be taken advantage of to functionalize molecules that already contain one or more nitrile functionalities. For example, the following reaction occurs in basic conditions:

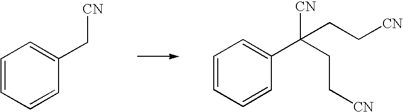

The cyanoethylation process typically requires a strong base as a catalyst. Most often such bases are alkali metal hydroxides such as, e.g., sodium oxide, lithium hydroxide, sodium hydroxide and potassium hydroxide. These metals, in turn, can exist as impurities in the amidoxime compound solution. The existence of such metals in the amidoxime compound solution is not acceptable for use in electronic, and more specifically, semiconductor manufacturing processes and as stabilizer for hydroxylamine free base and other radical sensitive reaction chemicals.

Exemplary alkali bases include, but are not limited to, metal ion free organic ammonium hydroxide compound, such as tetramethylammonium hydroxide, trimethylbenzylammonium hydroxide and the like.

All known water-soluble amidoxime compounds are generally suitable for inclusion in the composition and processes of the present invention. Of particular interest are those amidoxime compounds useful in the semiconductor industry such as, for example, those selected from the examples that follow. These exemplary amidoxime compounds also include a reaction pathway for their synthesis.

Nomenclatures are translated from chemical structures to their corresponding chemical names using ChemBioDraw Ultra from CambridgeSoft, MA. In the case of products from the reaction of sorbitol, the cyanoethylated sorbitol is named by its CAS# [2465-92-1] as 1,2,3,4,5,6-hexakis-O-(2-cyanoetyl)hexitol with a chemical formula of C₂₄H₃₂N₆O₆ and the corresponding amidoxime compound as 1,2,3,4,5,6-hexakis-O-[3-(hydroxyamino)-3-iminopropyl hexitol, CAS# [950752-25-7].

Abbreviations:

| | |
|---|---|
| Boiling point | Bp |
| Cat | Catalytic |
| Decomposed | Dec |
| Equivalent | eq |
| Ethanol | EtOH |
| Ether | Et₂O |
| Ethyl Acetate | EtOAc |
| Ethylenediamine tetracarboxylic acid | EDTA |
| Gram | g |
| Hydrochloride acid | HCl |
| Isopropyl Alcohol | iPrOH |
| Melting point | Mp |
| Methanol | MeOH |
| Methylene chloride | CH₂Cl₂ |
| Millimole or mole | Mmol or mol |
| Room temperature | Rt, RT |
| Tetramethylammonium hydroxide (25% in water) | TMAH |
| Trimethylbenzylammonium hydroxide (40% in MeOH) | Triton B |

Exemplary synthesis of amidoxime compounds from nitriles and cyanoethylated nitriles compounds.

Reactions to produce nitrile precursors to amidoxime compounds:

Cyanoethylation of diethylaminexine:

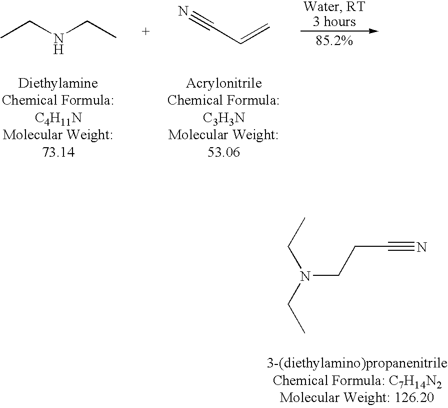

A solution of diethylamine (1 g, 13.67 mmol) and acrylonitrile (0.798 g, 15 mmol, 1.1 eq) in water (10 cm³) were stirred at room temperature for 3 hours, after which the mixture was extracted with dichloromethane (2×50 cm³). The organic extracts were evaporated under reduced pressure to give the pure cyanoethylated compound 3-(diethylamino)propanenitrile (1.47 g, 85.2%) as an oil.

Monocyanoethylation of glycine:

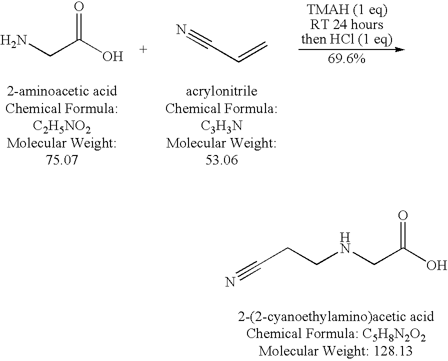

Glycine (5 g, 67 mmol) was suspended in water (10 cm³) and TMAH (25% in water, 24.3 g, 67 mmol) was added slowly, keeping the temperature at <30° C. with an ice-bath. The mixture was then cooled to 10° C. and acrylonitrile (3.89 g, 73 mmol) was added. The mixture was stirred overnight, and allowed to warm to room temperature slowly. The mixture was then neutralized with HCl (6M, 11.1 cm³), concentrated to 15 cm³ and diluted to 100 cm³ with EtOH. The solid precipitated was collected by filtration, dissolved in hot water (6 cm³) and reprecipitated with EtOH (13 cm³) to give 2-(2-cyanoethylamino)acetic acid (5.94 g, 69.6%) as a white solid, mp 192° C.; mp 190-191° C.).

Cyanoethylation of piperizine:

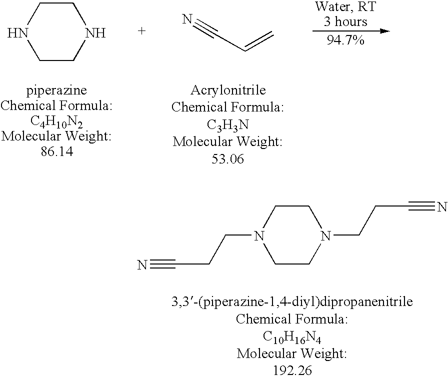

A solution of piperazine (1 g, 11.6 mmol) and acrylonitrile (1.6 g, 30.16 mmol, 2.6 eq) in water (10 cm³) were stirred at room temperature for 5 hours, after which the mixture was extracted with dichloromethane (2×50 cm³). The organic extracts were evaporated under reduced pressure to give the pure doubly cyanoethylated compound 3,3′-(piperazine-1,4-diyl)dipropanenitrile (2.14 g, 94.7%) as a white solid, mp 66-67° C.

Cyanoethylation of 2-ethoxyethanol:

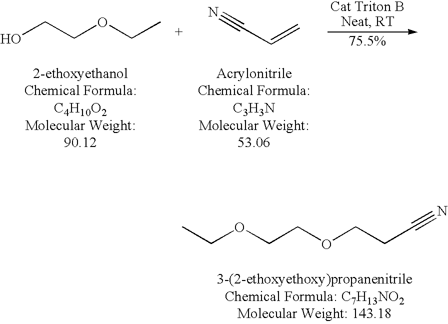

To an ice-water cooled mixture of 2-ethoxyethanol (1 g, 11.1 mmol) and Triton B (40% in MeOH, 0.138 g, 0.33 mmol) was added acrylonitrile (0.618 g, 11.6 mmol) and the mixture was stirred at room temperature for 24 hours. It was then neutralized with 0.1 M HCl (3.3 cm³) and extracted with CH₂Cl₂ (2×10 cm³) The extracts were concentrated under reduced pressure and the residue was Kugelrohr-distilled to give the product 3-(2-ethoxyethoxy)propanenitrile (1.20 g, 75.5%) as a colorless oil, bp 100-130° C./20 Torr.

Cyanoethylation of 2-(2-dimethylaminoethoxy)ethanol:

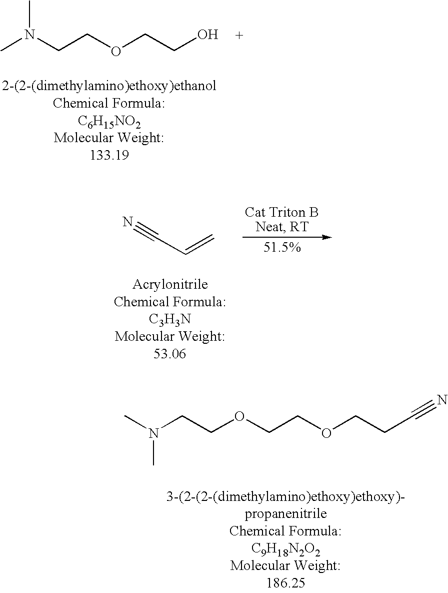

To an ice-water cooled mixture of 2-(2-dimethyleminothoxy)ethanol (1 g, 7.5 mmol) and Triton B (40% in MeOH, 0.094 g, 0.225 mmol) was added acrylonitrile (0.418 g, 7.9 mmol) and the mixture was stirred at room temperature for 24 hours. It was then neutralized with 0.1 M HCl (2.3 cm³) and extracted with CH₂Cl₂ (2×10 cm³) The extracts were concentrated under reduced pressure and the residue was purified by column chromatography (silica, Et₂O, 10% CH₂Cl₂, 0-10% EtOH) to give 3-(2-(2-(dimethylamino)ethoxy)ethoxy)propanenitrile as an oil.

Cyanoethylation of isobutyraldehyde:

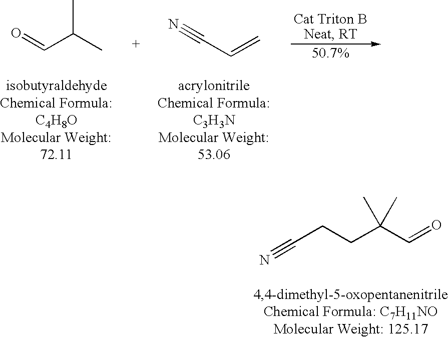

Isobutyraldehyde (1 g, 13.9 mmol) and acrylonitrile (0.81 g, 15 mmol) were mixed thoroughly and cooled with an ice-bath. Triton B (40% in MeOH, 0.58 g, 1.4 mmol) was added. The mixture was stirred at room temperature overnight. It was then neutralized with 0.1 M HCl (14 cm³) and extracted with CH₂Cl₂ (100 cm³) The extracts were concentrated under reduced pressure and the residue was Kugelrohr-distilled to give the product 4,4-dimethyl-5-oxopentanenitrile (0.8 g, 50.7%) as an oil, bp 125-130° C./20 Torr.

Cyanoethylation of aniline:

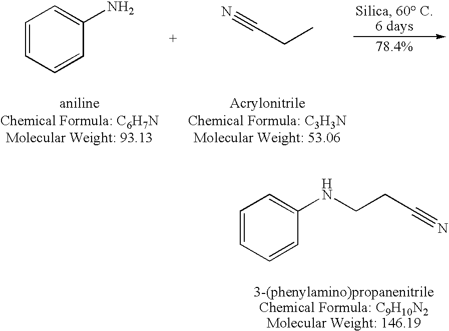

Silica was activated by heating it above 100° C. in vacuum and was then allowed to cool to room temperature under nitrogen. To the activated silica (10 g) was absorbed aniline (1.86 g, 20 mmol) and acrylonitrile (2.65 g, 50 mmol) and the flask was capped tightly. The contents were then stirred with a magnetic stirrer for 6 days at 60° C. After this time the mixture was cooled to room temperature and extracted with MeOH. The extracts were evaporated to dryness and the residue was Kugelrohr-distilled under high vacuum to give the product 3-(phenylamino)propanenitrile (2.29 g, 78.4%) as an oil which crystallised on standing; bp 120-150° C./1-2 Torr (lit bp 120° C./1 Torr), mp 50.5-52.5° C.

Cyanoethylation of ethylenediamine:

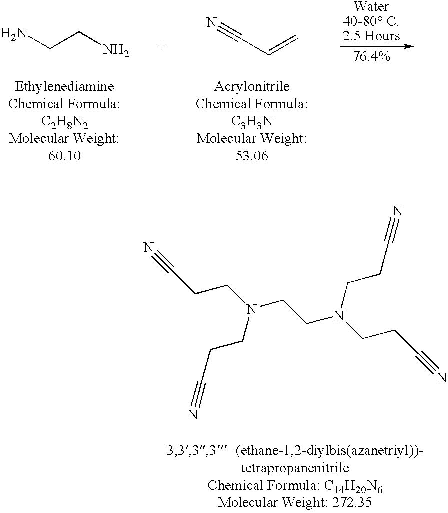

Acrylonitrile (110 g, 137 cm³, 2.08 mol) was added to a vigorously stirred mixture of ethylenediamine (25 g, 27.8 cm³, 0.416 mol) and water (294 cm³) at 40° C. over 30 min. During the addition, it was necessary to cool the mixture with a 25° C. water bath to maintain temperature at 40° C. The mixture was then stirred for additional 2 hours at 40° C. and 2 hours at 80° C. Excess acrylonitrile and half of the water were evaporated off and the residue, on cooling to room temperature, gave a white solid which was recrystallised from MeOH-water (9:1) to give pure product 3,3′,3″,3′″-(ethane-1,2-diylbis(azanetriyl))tetrapropanenitrile (86.6 g, 76.4%) as white crystals, mp 63-65° C.

Cyanoethylation of ethylene glycol:

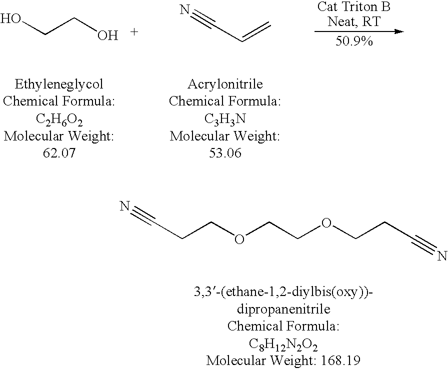

Small scale: Ethylene glycol (1 g, 16.1 mmol) was mixed with Triton B (40% in MeOH, 0.22 g 0.53 mmol) and cooled in an ice-bath while acrylonitrile (1.71 g, 32.2 mmol) was added. The mixture was stirred at room temperature for 60 hours after which it was neutralized with 0.1 M HCl (0.6 cm³) and extracted with CH₂Cl₂ (80 cm³) The extracts were concentrated under reduced pressure and the residue was Kugelrohr-distilled to give 3,3′-(ethane-1,2-diylbis(oxy))dipropanenitrile (1.08 g, 39.9%) as a light coloured oil, bp 150-170° C./20 Torr.

Large scale: Ethylene glycol (32.9 g, 0.53 mol) was mixed with Triton B (40% in MeOH, 2.22 g, 5.3 mmol) and cooled in an ice-bath while acrylonitrile (76.2 g, 1.44 mol) was added. The mixture was allowed to warm slowly to room temperature and stirred for 60 hours after which it was neutralized with 0.1 M HCl (50 cm³) and extracted with CH₂Cl₂ (300 cm³) The extracts were passed through a silica plug three times to reduce the brown colouring to give 86 g (quantitative yield) of the product as an amber coloured oil, pure by ¹H-NMR, containing 10 g of water (total weight 96 g, amount of water calculated by ¹H NMR integral sizes).

Cyanoethylation of diethyl malonate:

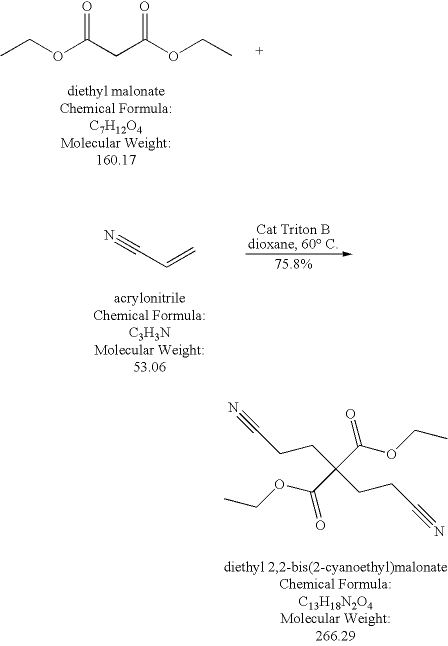

To a solution of diethyl malonate (1 g, 6.2 mmol) and Triton B (40% in MeOH, 0.13 g, 0.31 mmol) in dioxane (1.2 cm³) was added dropwise acrylonitrile (0.658 g, 12.4 mmol) and the mixture was stirred at 60° C. overnight. The mixture was then cooled to room temperature and neutralized with 0.1 M HCl (3 cm³) and poured to ice-water (10 cm³). Crystals precipitated during 30 min. These were collected by filtration and recrystallised from EtOH (cooling in freezer before filtering off) to give diethyl 2,2-bis(2-cyanoethyl)malonate (1.25 g, 75.8%) as a white solid, mp 62.2-63.5° C.

Hydrolysis of diethyl 2,2-bis(2-cyanoethyl)malonate:

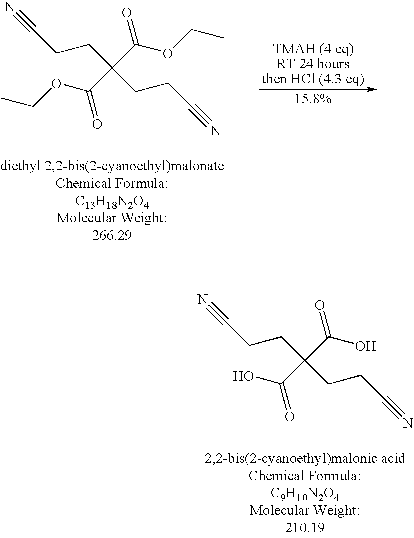

Diethyl 2,2-bis(2-cyanoethyl)malonate (2 g, 7.51 mmol) was added to TMAH (25% in water, 10.95 g, 30.04 mmol) at room temperature. The mixture was stirred for 24 hours, and was then cooled to 0° C. A mixture of 12M HCl (2.69 cm³, 32.1 mmol) and ice (3 g) was added and the mixture was extracted with CH₂Cl₂ (5×50 cm³). The extracts were evaporated under vacuum to give 2,2-bis(2-cyanoethyl)malonic acid (0.25 g, 15.8%) as a colourless very viscous oil (lit decomposed. 158° C.).

Dicyanoethylation of glycine to give 2-(bis(2-cyanoethyl)amino)acetic acid:

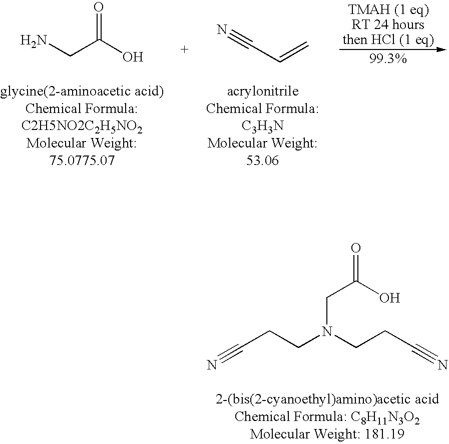

Glycine (5 g, 67 mmol) was suspended in water (10 cm³) and TMAH (25% in water, 24.3 g, 67 mmol) was added slowly, keeping the temperature at <30° C. with an ice-bath. The mixture was then cooled to 10° C. and acrylonitrile (7.78 g, 146 mmol) was added. The mixture was stirred overnight, and allowed to warm to room temperature slowly. It was then heated at 50° C. for 2 hours, using a reflux condenser. After cooling with ice, the mixture was neutralized with HCl (6M, 11.1 cm³) and concentrated to a viscous oil. This was dissolved in acetone (1 cm³) and filtered to remove NMe₄Cl. The filtrate was concentrated under reduced pressure to give an oil that was treated once more with acetone (100 cm³) and filtered to remove more NMe₄Cl. Concentration of the filtrate gave 2-(bis(2-cyanoethyl)amino)acetic acid (11.99 g, 99.3%) as a colourless, viscous oil that crystallised over 1 week at room temperature to give a solid product, mp 73° C. (lit mp 77.8-78.8° C. Duplicate ¹³C signals indicate a partly zwitterionic form in CDCl₃ solution.

When NaOH is used in the literature procedure, the NaCl formed is easier to remove and only one acetone treatment is necessary.

Dicyanoethylation of N-methyldiethanolamine to give 3,3′-(2,2′-(methylazanediyl)bis(ethane-2,1-diyl)bis(oxy))dipropanenitrile:

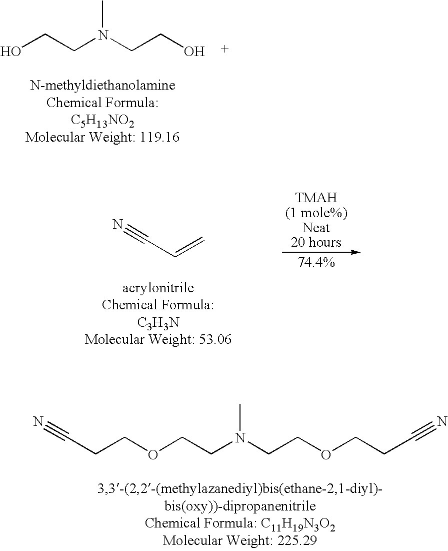

To a cooled, stirred mixture of N-methyldiethanolamine (2 g, 17 mmol) and acrylonitrile (2.33 g, 42 mmol) was added TMAH (25% in water, 0.25 cm³, 0.254 g, 7 mmol). The mixture was then stirred overnight, and allowed to warm to room temperature slowly. It was then filtered through silica using a mixture of Et₂O and CH₂Cl₂ (1:1, 250 cm³) and the filtrated was evaporated under reduced pressure to give 3,3′-(2,2′-(methylazanediyl)bis(ethane-2,1-diyl)bis(oxy))dipropanenitrile (2.85 g, 74.4%) as a colourless oil.

Dicyanoethylation of glycine anhydride:

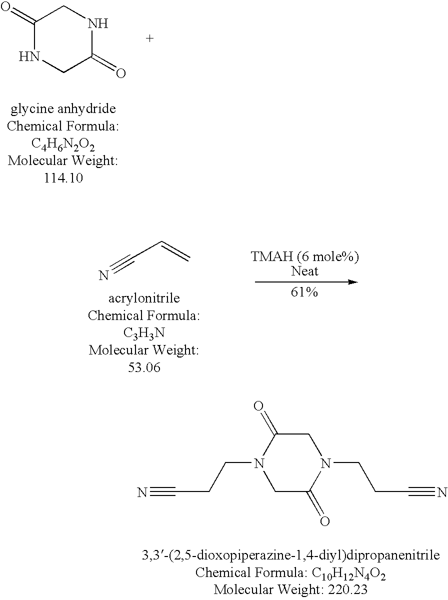

Glycine anhydride (2 g, 17.5 mmol) was mixed with acrylonitrile (2.015 g, 38 mmol) at 0° C. and TMAH (25% in water, 0.1 cm³, 0.1 g, 2.7 mmol) was added. The mixture was then stirred overnight, allowing it to warm to room temperature slowly. The solid formed was recrystallised from EtOH to give 3,3′-(2,5-dioxopiperazine-1,4-diyl)dipropanenitrile (2.35 g, 61%) as a white solid, mp 171-173° C. (lit mp 166° C.).

N,N-Dicyanoethylation of acetamide:

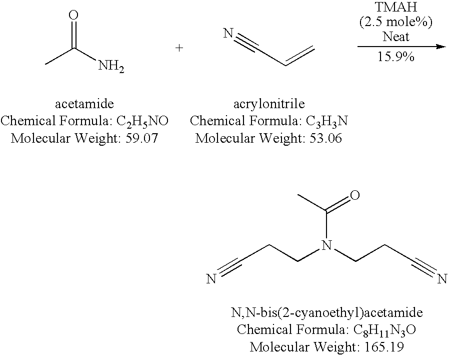

Acetamide (2 g, 33.9 mmol) was mixed with acrylonitrile (2.26 g, 42.7 mmol) at 0° C. and TMAH (25% in water, 0.06 cm³, 0.06 g, 1.7 mmol) was added. The mixture was then stirred overnight, allowing it to warm to room temperature slowly. The mixture was filtered through a pad of silica with the aid of Et₂O/CH₂Cl₂ (200 cm³) and the filtrate was concentrated under reduced pressure. The product was heated with spinning in a Kugelrohr at 150° C./2 mmHg to remove side products and to give N,N-bis(2-cyanoethyl)acetamide (0.89 g, 15.9%) as a viscous oil.

The N-substituent in the amides is non-equivalent due to amide rotation.

Tricyanoethylation of ammonia:

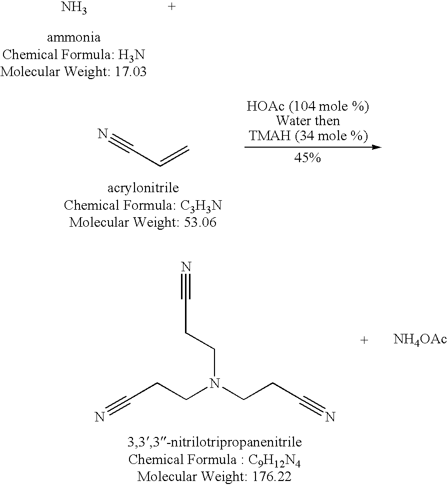

Ammonia (aq 35%, 4.29, 88 mmol) was added dropwise to ice-cooled AcOH (5.5 g, 91.6 mmol) in water (9.75 cm³), followed by acrylonitrile (4.65 g, 87.6 mol). The mixture was stirred under reflux for 3 days, after which it was cooled with ice and aq. TMAH (25% in water, 10.94 g, 30 mmol) was added. The mixture was kept cooled with ice for 1 hour. The crystals formed was collected by filtration and washed with water. The product was dried in high vacuum to give 3,3′,3″-nitrilotripropanenitrile (2.36 g, 45.8%) as a white solid, mp 59-61° C. (lit mp 59° C.).

When NaOH was used to neutralise the reaction (literature procedure), the yield was higher, 54.4%.

Dicyanoethylation of cyanoacetamide:

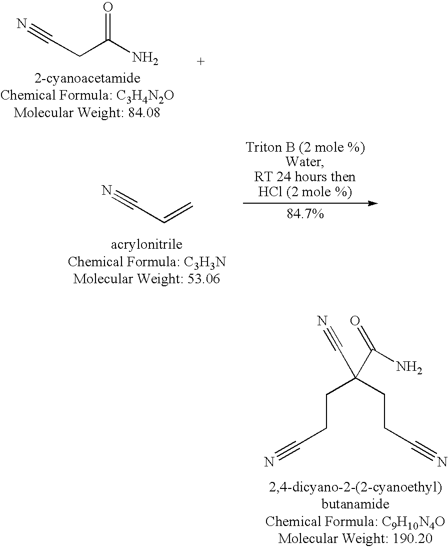

To a stirred mixture of cyanoacetamide (2.52 g, 29.7 mmol) and Triton B (40% in MeOH, 0.3 g, 0.7 mmol) in water (5 cm³) was added acrylonitrile (3.18 g, 59.9 mmol) over 30 minutes with cooling. The mixture was then stirred at room temperature for 30 min and then allowed to stand for 1 hour. EtOH (20 g) and 1M HCl (0.7 cm³) were added and the mixture was heated until all solid had dissolved. Cooling to room temperature gave crystals that were collected by filtration and recrystallised from EtOH to give 2,4-dicyano-2-(2-cyanoethyl)butanamide (4.8 g, 84.7%) as a pale yellow solid, mp 118-120° C.

N,N-Dicyanoethylation of anthranilonitrile:

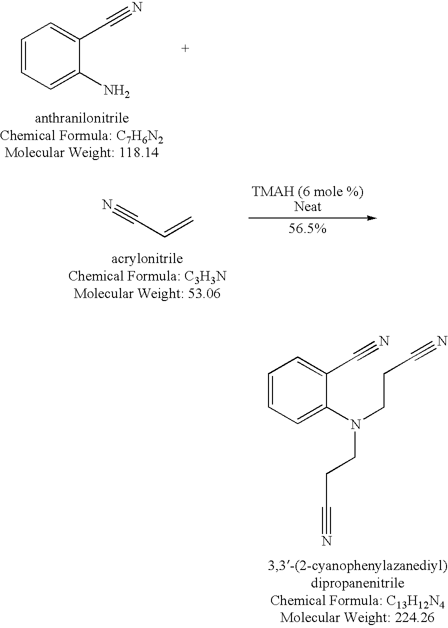

Anthranilonitrile (2 g, 16.9 mmol) was mixed with acrylonitrile (2.015 g, 38 mmol) at 0° C. and TMAH (25% in water, 0.1 cm³, 0.1 g, 2.7 mmol) was added. The mixture was then stirred overnight, allowing it to warm to room temperature slowly. The product was dissolved in CH₂Cl₂ and filtered through silica using a mixture of Et₂O and CH₂Cl₂ (1:1, 250 cm³). The filtrate was evaporated to dryness and the solid product was recrystallised from EtOH (5 cm³) to give 3,3′-(2-cyanophenylazanediyl)dipropanenitrile (2.14 g, 56.5%) as an off-white solid, mp 79-82° C.

Dicyanoethylation of malononitrile:

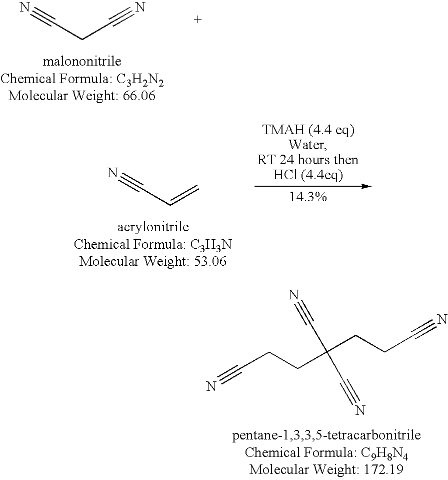

Malononitrile (5 g, 75.7 mmol) was dissolved in dioxane (10 cm³), followed by trimethylbenzylammonium hydroxide (Triton B, 40% in MeOH, 1.38 g, 3.3 mmol). The mixture was cooled while acrylonitrile (8.3 g, 156 mmol) was added. The mixture was stirred overnight, allowing it to warm to room temperature slowly. It was then neutralized with HCl (1 M, 3.3 cm³) and poured into ice-water. The mixture was extracted with CH₂Cl₂ (200 cm³) and the extracts were evaporated under reduced pressure. The product was purified by column chromatography (silica, 1:1 EtOAc-petroleum) followed by recrystallisation to give 1,3,3,5-tetracarbonitrile (1.86 g, 14.3%), mp 90-92° C. (lit mp 92° C.).

Tetracyanoethylation of pentaerythritol:

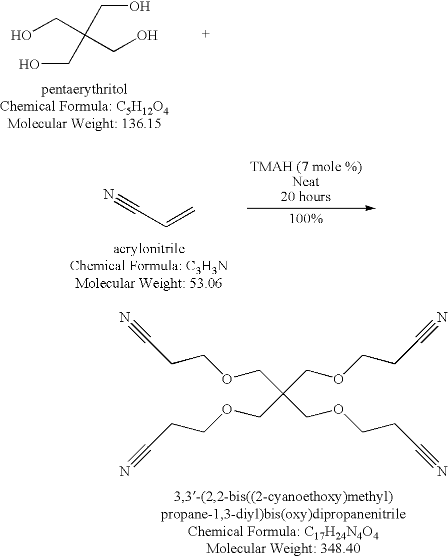

Pentaerythritol (2 g, 14.7 mmol) was mixed with acrylonitrile (5 cm³, 4.03 g, 76 mmol) and the mixture was cooled in an ice-bath while tetramethylammonium hydroxide (=TMAH, 25% in water, 0.25 cm³, 0.254 g, 7 mmol) was added. The mixture was then stirred at room temperature for 20 hours. After the reaction time the mixture was filtered through silica using a mixture of Et₂O and CH₂Cl₂ (1:1, 250 cm³) and the filtrated was evaporated under reduced pressure to give 3,3′-(2,2-bis((2-cyanoethoxy)methyl)propane-1,3-diyl)bis(oxy)dipropanenitrile (5.12 g, 100%) as a colourless oil.

Hexacyanoethylation of sorbitol:

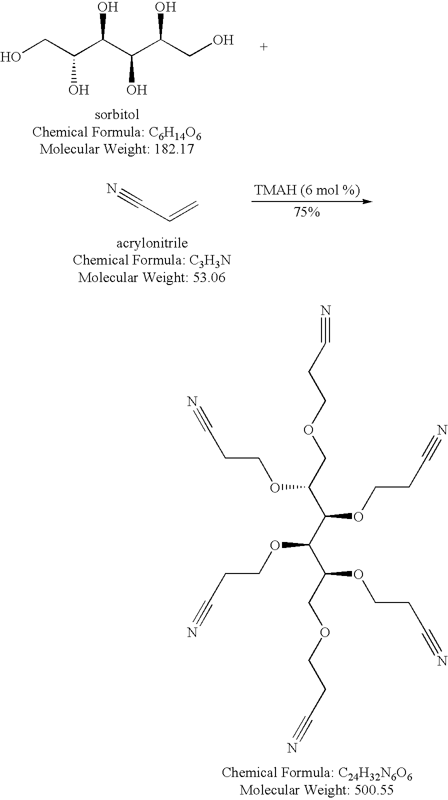

Sorbitol (2 g, 11 mmol) was mixed with acrylonitrile (7 cm³, 5.64 g, 106 mmol) and the mixture was cooled in an ice-bath while tetramethylammonium hydroxide (=TMAH, 25% in water, 0.25 cm³, 0.254 g, 7 mmol) was added. The mixture was then stirred at room temperature for 48 hours, adding another 0.25 cm³ of TMAH after 24 hours. After the reaction time the mixture was filtered through silica using a mixture of Et₂O and CH₂Cl₂ (1:1, 250 cm³) and the filtrate was evaporated under reduced pressure to give a fully cyanoethylated product (4.12 g, 75%) as a colourless oil.

Tricyanoethylation of diethanolamine to give 3,3′-(2,2′-(2-cyanoethylazanediyl)bis(ethane-2,1-diyl)bis(oxy))dipropanenitrile:

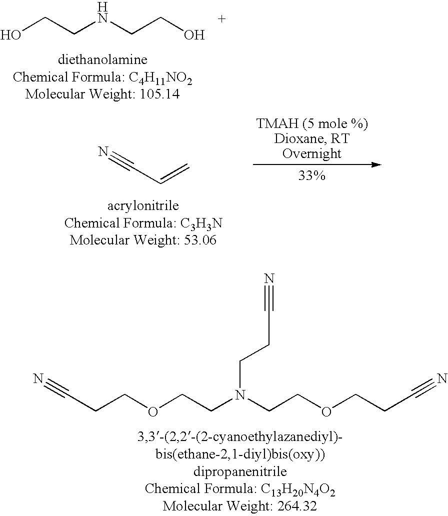

To an ice-cooled stirred solution of diethanolamine (2 g, 19 mmol) and TMAH (25% in water, 0.34 cm³, 0.35 g, 9.5 mmol) in dioxane (5 cm³) was added acrylonitrile (3.53 g, 66.1 mmol) dropwise. The mixture was then stirred overnight, and allowed to warm to room temperature. More acrylonitrile (1.51 g, 28 mmol) and TMAH (0.25 cm³, 7 mmol) was added and stirring was continued for additional 24 h. The crude mixture was filtered through a pad of silica (Et₂O/CH₂Cl₂ as eluent) and evaporated to remove dioxane. The residue was purified by column chromatography (silica, Et₂O to remove impurities followed by EtOAc to elute product) to give 3,3′-(2,2′-(2-cyanoethylazanediyl)bis(ethane-2,1-diyl)bis(oxy))dipropanenitrile (1.67 g, 33%) as an oil.

Reactions to produce amidoxime compounds

Reaction of acetonitrile to give N′-hydroxyacetimidamide:

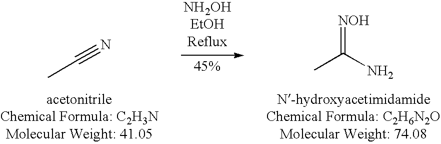

A solution of acetonitrile (0.78 g, 19 mmol) and hydroxylamine (50% in water, 4.65 cm³, 5.02 g, 76 mmol, 4 eq) in EtOH (100 cm³) was stirred under reflux for 1 hours, after which the solvent was removed under reduced pressure and the residue was recrystallised from iPrOH to give the product N′-hydroxyacetimidamide (0.63 g, 45%) as a solid, mp 134.5-136.5° C.

Reaction of octanonitrile to give N′-hydroxyoctanimidamide:

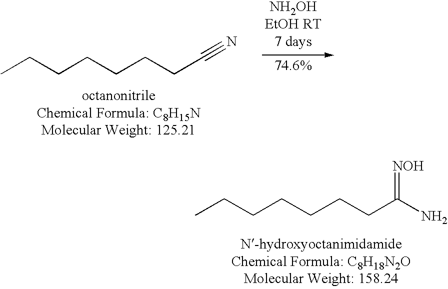

Octanonitrile (1 g, 7.99 mmol) and hydroxylamine (50% in water, 0.74 cm3, 0.79 g, 12 mmol 1.5 eq) in EtOH (1 cm³) were stirred at room temperature for 7 days. Water (10 cm³) was then added. This caused crystals to precipitate, these were collected by filtration and dried in high vacuum line to give the product N′-hydroxyoctanimidamide (0.94 g, 74.6%) as a white solid, mp 73-75° C.

Reaction of chloroacetonitrile to give 2-chloro-N′-hydroxyacetimidamide:

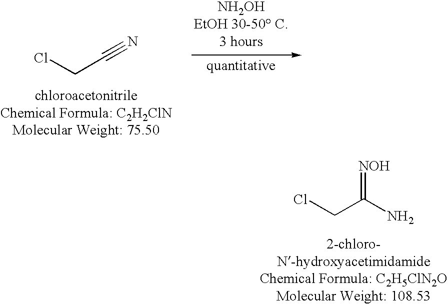

Chloroacetonitrile (1 g, 13 mmol) and hydroxylamine (50% in water, 0.89 cm³, 0.96 g, 14.6 mmol, 1.1 eq) in EtOH (1 cm³) were stirred at 30-50° C. for 30 min. The mixture was then extracted with Et2O (3×50 cm³). The extracts were evaporated under reduced pressure to give the product 2-chloro-N′-hydroxyacetimidamide (0.81 g, 57.4%) as a yellow solid, mp 79-80° C.

Reaction of ethyl 2-cyanoacetate to give 3-amino-N-hydroxy-3-(hydroxyimino)propanamide:

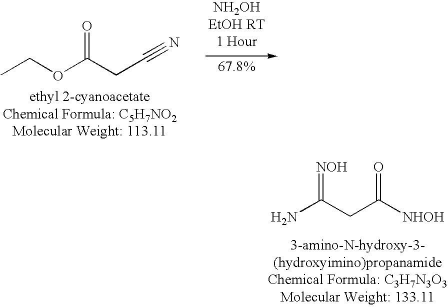

Ethyl cyanoacetate (1 g, 8.84 mmol) and hydroxylamine (50% in water, 1.19 cm³, 1.29 g, 19.4 mmol, 2.2 eq) in EtOH (1 cm³) were allowed to stand at room temperature for 1 hour with occasional swirling. The crystals formed were collected by filtration and dried in high vacuum line to give a colorless solid, 3-amino-N-hydroxy-3-(hydroxyimino)propanamide, mp 158° C. (decomposed) (lit mp 150° C.).

Reaction of 3-hydroxypropionitrile to give N′,3-dihydroxypropanimidamide:

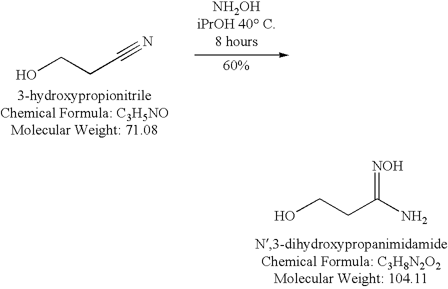

Equal molar mixture of 3-hydroxypropionitrile and hydroxylamine heated to 40° C. for 8 hours with stirring. The solution is allowed to stand overnight yielding a fine slightly off white precipitate. The precipitated solid was filtered off and washed with iPrOH and dried to a fine pure white crystalline solid N′,3-dihydroxypropanimidamide mp 94° C.

Reaction of 2-cyanoacetic acid to give isomers of 3-amino-3-(hydroxyimino)propanoic acid:

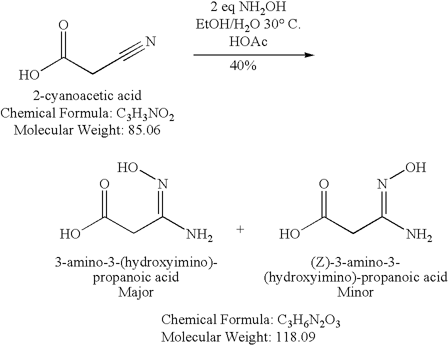

2-Cyanoacetic acid (1 g, 11.8 mmol) was dissolved in EtOH (10 cm³) and hydroxylamine (50% in water, 0.79 cm3, 0.85 g, 12.9 mmol, 1.1 eq) was added. The mixture was warmed at 40° C. for 30 min and the crystals formed (hydroxylammonium cyanoacetate) were filtered off and dissolved in water (5 cm³). Additional hydroxylamine (50% in water, 0.79 cm3, 0.85 g, 12.9 mmol, 1.1 eq) was added and the mixture was stirred at room temperature overnight. Acetic acid (3 cm³) was added and the mixture was allowed to stand for a few hours. The precipitated solid was filtered off and dried in high vacuum line to give the product 3-amino-3-(hydroxyimino)propanoic acid (0.56 g, 40%) as a white solid, mp 136.5° C. (lit 144° C.) as two isomers.

Characterization of the product using FTIR and NMR are as follows: vmax(KBr)/cm−1 3500-3000 (br), 3188, 2764, 1691, 1551, 1395, 1356, 1265 and 1076; δH (300 MHz; DMSO-d6; Me4Si) 10.0-9.0 (br, NOH and COOH), 5.47 (2H, br s, NH2) and 2.93 (2H, s, CH2); δC (75 MHz; DMSO-d6; Me4Si) 170.5 (COOH minor isomer), 170.2 (COOH major isomer), 152.8 (C(NOH)NH2 major isomer) 148.0 (C(NOH)NH2 minor isomer), 37.0 (CH2 minor isomer) and 34.8 (CH2 major isomer).

Reaction of adiponitrile to give N′1,N′6-dihydroxyadipimidamide:

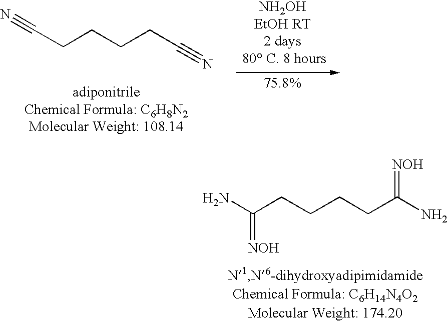

Adiponitrile (1 g, 9 mmol) and hydroxylamine (50% in water, 1.24 cm3, 1.34 g, 20 mmol, 2.2 eq) in EtOH (10 cm3) were stirred at room temperature for 2 days and then at 80° C. for 8 hours. The mixture was allowed to cool and the precipitated crystals were collected by filtration and dried in high vacuum line to give the product N′1,N′6-dihydroxyadipimidamide (1.19 g, 75.8%) as a white solid, mp 160.5 (decomposed) (lit decomposed 168-170° C.

Reaction of sebaconitrile to give N′1,N′10-dihydroxydecanebis(imidamide):

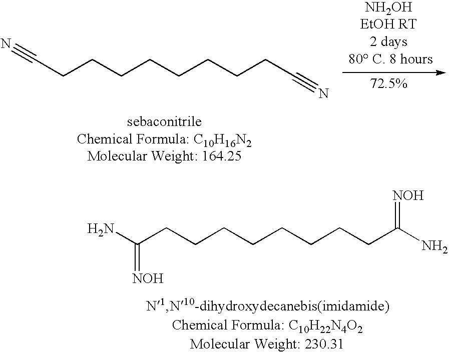

Sebaconitrile (1 g, 6 mmol) and hydroxylamine (50% in water, 0.85 cm³, 0.88 g, 13.4 mmol, 2.2 eq) in EtOH (12 cm³) were stirred at room temperature for 2 days and then at 80° C. for 8 h. The mixture was allowed to cool and the precipitated crystals were collected by filtration and dried in high vacuum line to give the product N′1,N′10-dihydroxydecanebis(imidamide) (1 g, 72.5%); mp 182° C.

Reaction of 2-cyanoacetamide to give 3-amino-3-(hydroxyimino)propanamide:

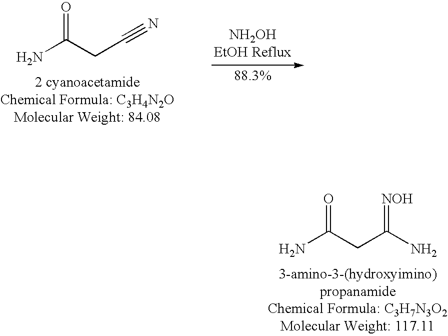

2-Cyanoacetamide (1 g, 11.9 mmol) and hydroxylamine (0.8 cm³, 13 mmol, 1.1 eq) in EtOH (6 cm³) were stirred under reflux for 2.5 hours. The solvents were removed under reduced pressure and the residue was washed with CH₂Cl₂ to give the product 3-amino-3-(hydroxyimino)propanamide (1.23 g, 88.3%) as a white solid, mp 159° C.

Reaction of glycolonitrile to give N′,2-dihydroxyacetimidamide:

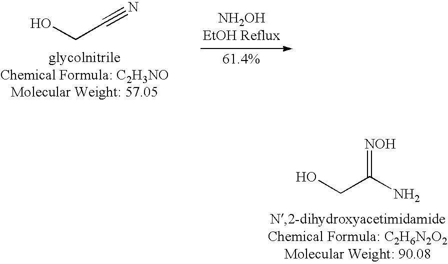

Glycolonitrile (1 g, 17.5 mmol) and hydroxylamine (50% in water, 2.15 cm³, 35 mmol, 2 eq) in EtOH (10 cm³) were stirred under reflux for 6 hours and then at room temperature for 24 hours. The solvent was evaporated and the residue was purified by column chromatography (silica, 1:3 EtOH—CH₂Cl₂) to give the product N′,2-dihydroxyacetimidamide (0.967 g, 61.4%) as an off-white solid, mp 63-65° C.

Reaction of 5-hexynenitrile to give 4-cyano-N′-hydroxybutanimidamide:

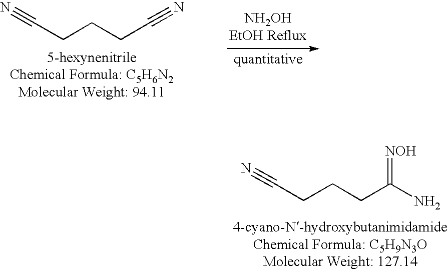

A solution of 5-hexynenitrile (0.93 g 10 mmol) and hydroxylamine (50% in water, 1.22 cm³, 20 mmol) was stirred under reflux for 10 hours, after which volatiles were removed under reduced pressure to give the product 4-cyano-N′-hydroxybutanimidamide (1.30 g, 100%) as a white solid, mp 99.5-101° C.

Reaction of iminodiacetonitrile to give 2,2′-azanediylbis(N′-hydroxyacetimidamide:

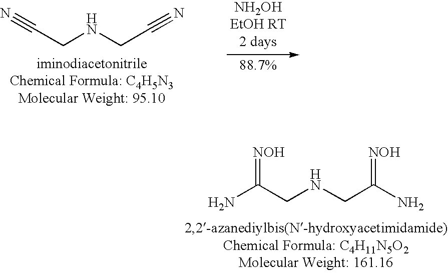

Commercial iminodiacetonitrile (Alfa-Aesar) was purified by dispersing the compound in water and extracting with dichloromethane, then evaporating the organic solvent from the extracts to give a white solid. Purified iminodiacetonitrile (0.82 g) and hydroxylamine (50% in water, 2.12 ml, 2.28 g, 34.5 mmol, 4 eq) in MeOH (6.9 ml) and water (6.8 ml) were stirred at room temperature for 48 hours. Evaporation of volatiles under reduced pressure gave a colorless liquid which was triturated with EtOH (40° C.) to give 2,2′-azanediylbis(N′-hydroxyacetimidamide) (1.23 g, 88.7%) as a white solid, mp 135-136° C., (lit mp 138° C.).

Reaction of 3-methylaminopropionitrile to give N′-hydroxy-3-(methylamino)propanimidamide:

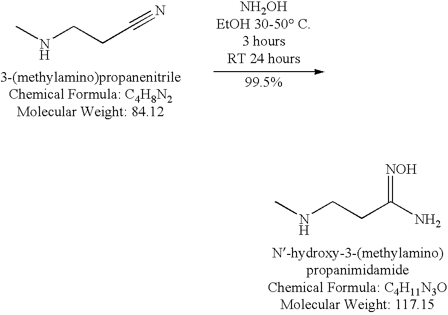

A solution of 3-methylaminopropionitrile (1 g, 11.9 mmol) and hydroxylamine (50% in water, 0.8 cm3, 0.864 g, 13.1 mmol, 1.1 eq) in EtOH (1 cm³) was stirred at 30-50° C. for 3 hours and then at room temperature overnight. The solvent was removed under reduced pressure (rotary evaporator followed by high vacuum line) to give the product N′-hydroxy-3-(methylamino)propanimidamide (1.387 g, 99.5%) as a thick pale yellow oil.

Reaction of 3-(diethylamino)propanenitrile to give 3-(diethylamino)-N′-hydroxypropanimidamide:

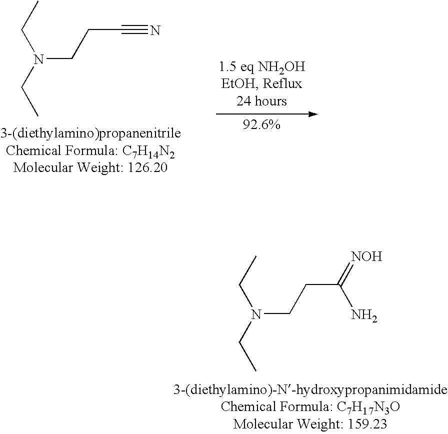

A solution of 3-(diethylamino)propanenitrile (1 g, 8 mmol) and NH₂OH (50% in water, 0.73 cm³, 11.9 mmol) in EtOH (10 cm³) were heated to reflux for 24 hours, after which the solvent and excess hydroxylamine were removed by rotary evaporator. The residue was freeze-dried and kept in high vacuum line until it slowly solidified to give give 3-(diethylamino)-N′-hydroxypropanimidamide (1.18 g, 92.6%) as a white solid, mp 52-54° C.

Reaction of 3,3′,3″-nitrilotripropanenitrile with hydroxylamine to give 3,3′,3″-nitrilotris(N′-hydroxypropanimidamide):

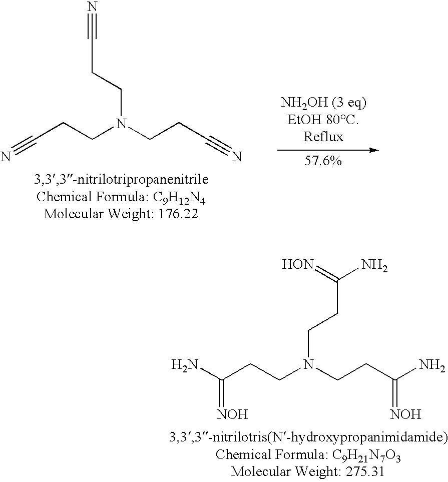

A solution of 3,3′,3″-nitrilotripropanenitrile (2 g, 11.35 mmol) and hydroxylamine (50% in water, 2.25 g, 34 mmol) in EtOH (25 cm³) was stirred at 80° C. overnight, then at room temperature for 24 hours. The white precipitate was collected by filtration and dried in high vacuum to give 3,3′,3″-nitrilotris(N′-hydroxypropanimidamide) (1.80 g, 57.6%) as a white crystalline solid, mp 195-197° C. (decomposed)

Reaction of 3-(2-ethoxyethoxy)propanenitrile to give 3-(2-ethoxyethoxy)-N′-hydroxypropanimidamide:

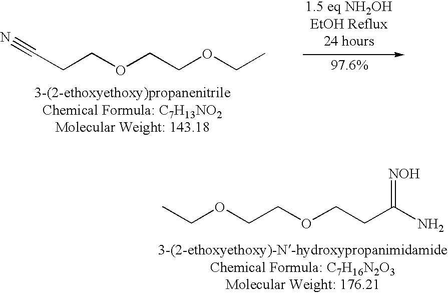

A solution of 3-(2-ethoxyethoxy)propanenitrile (1 g, 7 mmol) and NH₂OH (50% in water, 0.64 cm³, 10.5 mmol) in EtOH (10 cm³) were heated to reflux for 24 hours, after which the solvent and excess hydroxylamine were removed by rotary evaporator. The residue was freeze-dried and kept in high vacuum line for several hours to give 3-(2-ethoxyethoxy)-N′-hydroxypropanimidamide (1.2 g, 97.6%) as a colourless oil.

Reaction of 3-(2-(2-(dimethylamino)ethoxy)ethoxy)propanenitrile to give 3-(2-(2-(dimethylamino)ethoxy)ethoxy)-N′-hydroxypropanimidamide:

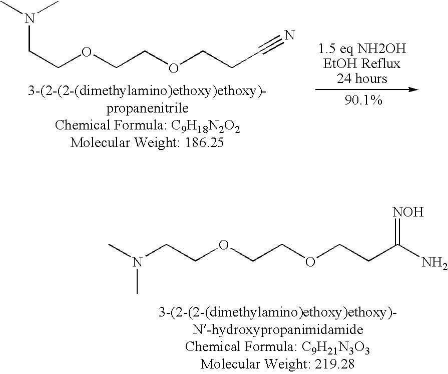

A solution of 3-(2-(2-(dimethylamino)ethoxy)ethoxy)propanenitrile (0.5 g, 2.68 mmol) and NH₂OH (50% in water, 0.25 cm³, 4 mmol) in EtOH (10 cm³) were stirred at 80° C. for 24 hours, after which the solvent and excess hydroxylamine were removed by rotary evaporator. The residue was freeze-dried and kept in high vacuum line for several hours to give 3-(2-(2-(dimethylamino)ethoxy)ethoxy)-N′-hydroxypropanimidamide (0.53 g, 90.1%) as a light yellow oil.

Reaction of 3,3′-(2,2′-(2-cyanoethylazanediyl)bis(ethane-2,1-diyl)bis(oxy))dipropanenitrile with hydroxylamine to give 3,3′-(2,2′-(3-amino-3-(hydroxyimino)propylazanediyl)bis(ethane-2,1-diyl))bis(oxy)bis(N′-hydroxypropanimidamide):

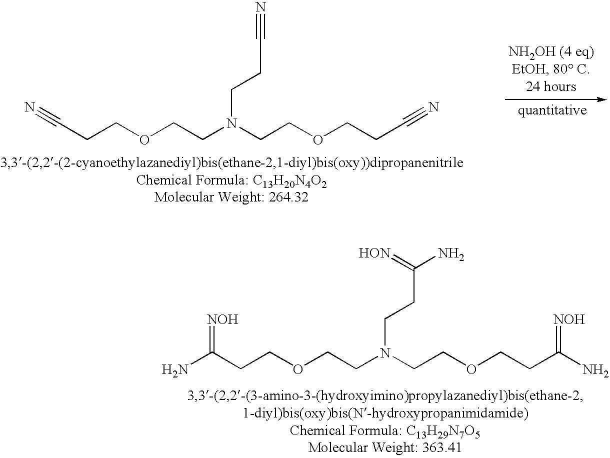

Treatment of 3,3′-(2,2′-(2-cyanoethylazanediyl)bis(ethane-2,1-diyl)bis(oxy))dipropanenitrile (0.8 g, 3 mmol) with NH₂OH (0.74 cm³, 12.1 mmol) in EtOH (8 cm³) gave 3,3′-(2,2′-(3-amino-3-(hydroxyimino)propylazanediyl)bis(ethane-2,1-diyl))bis(oxy)bis(N′-hydroxypropanimidamide) (1.09 g, 100%) as an oil.

Reaction of iminodipropionitrile to give 3,3′-azanediylbis(N′-hydroxypropanimidamide):

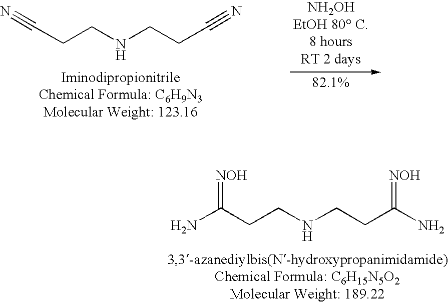

Iminodipropionitrile (1 g, 8 mmol) and hydroxylamine (50% in water, 1 cm³, 1.07 g, 16 mmol, 2 eq) in EtOH (8 cm³) were stirred at room temperature for 2 days and then at 80° C. for 8 hours. The mixture was allowed to cool and the precipitated crystals were collected by filtration and dried in high vacuum line to give the product 3,3′-azanediylbis(N′-hydroxypropanimidamide) (1.24 g, 82.1%) as a white solid, mp 180° C. (lit 160° C.).

Reaction of 3,3′,3″,3′″-(ethane-1,2-diylbis(azanetriyl))tetrapropanenitrile to give 3,3′,3″,3′″-(ethane-1,2-diylbis(azanetiyl))tetrakis(N′-hydroxypropanimidamide) to produce EDTA analog:

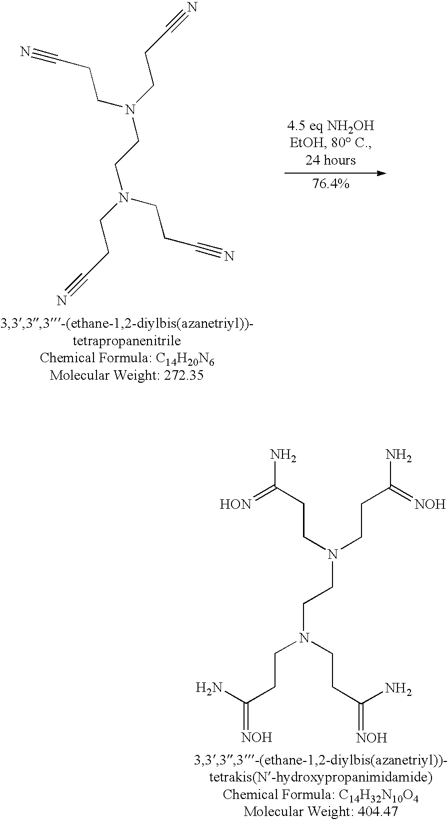

A solution of 3,3′,3″,3′″-(ethane-1,2-diylbis(azanetriyl))tetrapropanenitrile (1 g, 4 mmol) and NH₂OH (50% in water, 1.1 cm³, 18.1 mmol) in EtOH (10 cm³) was stirred at 80° C. for 24 hours and was then allowed to cool to room temperature. The solid formed was collected by filtration and dried under vacuum to give 3,3′,3″,3′″-(ethane-1,2-diylbis(azanetriyl))tetrakis(N′-hydroxypropanimidamide) (1.17 g, 76.4%) as a white solid, mp 191-192° C.

Reaction of 3,3′-(2,2-bis((2-cyanoethoxy)methyl)propane-1,3-diyl)bis(oxy)dipropanenitrile with hydroxylamine to give 3,3′-(2,2-bis((3-(hydroxyamino)-3-iminopropoxy)methyl)propane-1,3-diyl)bis(oxy)bis(N-hydroxypropanimidamide):

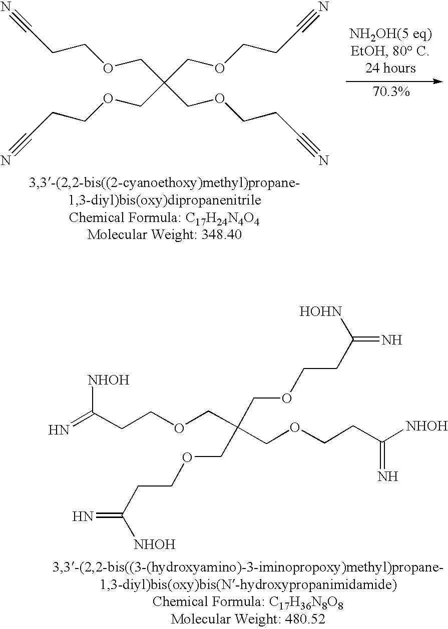

To a solution of 3,3′-(2,2-bis((2-cyanoethoxy)methyl)propane-1,3-diyl)bis(oxy)dipropanenitrile (1 g 2.9 mmol) in EtOH (10 ml) was added NH2OH (50% in water, 0.88 ml, 0.948 g, 14.4 mmol), the mixture was stirred at 80° C. for 24 hours and was then cooled to room temperature. Evaporation of the solvent and excess NH2OH in the rotary evaporator followed by high vacuum for 12 hours gave 3,3′-(2,2-bis((3-(hydroxyamino)-3-iminopropoxy)methyl)propane-1,3-diyl)bis(oxy)bis(N-hydroxypropanimidamide) (0.98 g, 70.3%) as a white solid, mp 60° C.;

Reaction of 3,3′-(2-cyanophenylazanediyl)dipropanenitrile with hydroxylamine to give 3,3′-(2-(N′-hydroxycarbamimidoyl)phenylazanediyl)bis(N′-hydroxypropanimidamide):

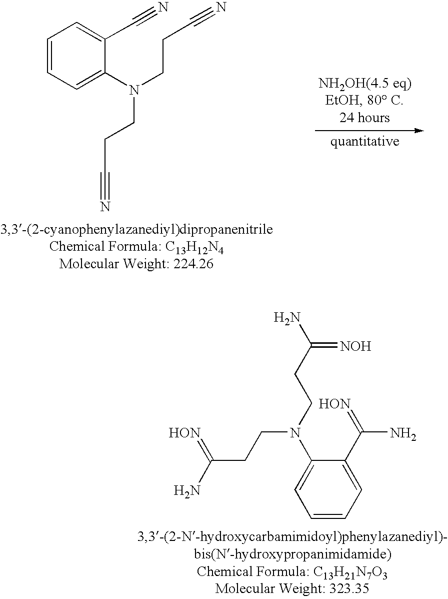

Treatment of 3,3′-(2-cyanophenylazanediyl)dipropanenitrile (1 g, 4.46 mmol) with NH2OH (1.23 ml, 20 mmol) in EtOH (10 ml) gave a crude product that was triturated with CH₂Cl₂ to give 3,3′-(2-(N′-hydroxycarbamimidoyl)phenylazanediyl)bis(N′-hydroxypropanimidamide) (1.44 g, 100%) as a solid, decomposed. 81° C.

Reaction of N,N-bis(2-cyanoethyl)acetamide with hydroxylamine to give N,N-bis(3-amino-3-(hydroxyimino)propyl)acetamide:

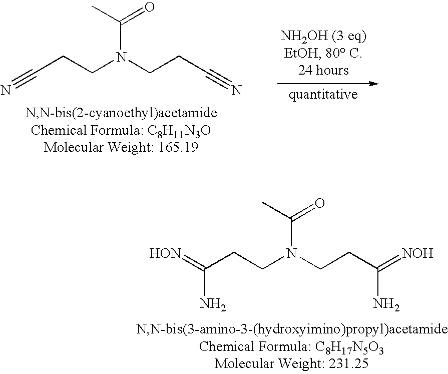

Treatment of N,N-bis(2-cyanoethyl)acetamide (0.5 g, 3.03 mmol) with NH₂OH (0.56 ml, 9.1 mmol) in EtOH (5 ml) gave N,N-bis(3-amino-3-(hydroxyimino)propyl)acetamide (0.564 g, 100%) as a white solid, mp 56.4-58° C.

Reaction of 3,3′-(2,2′-(methylazanediyl)bis(ethane-2,1-diyl)bis(oxy))dipropanenitrile with hydroxylamine to give 3,3′-(2,2′-(methylazanediyl)bis(ethane-2,1-diyl)bis(oxy))bis(N′-hydroxypropanimidamide);

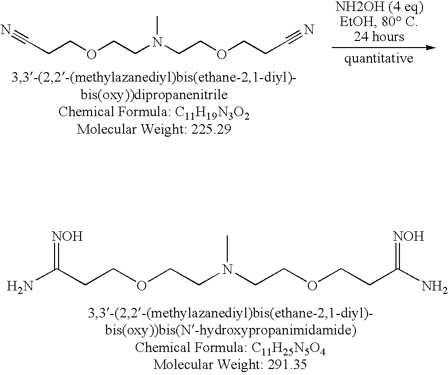

Treatment of 3,3′-(2,2′-(methylazanediyl)bis(ethane-2,1-diyl)bis(oxy))dipropanenitrile (1 g, 4.4 mmol) with NH₂OH (0.82 ml, 13.3 mmol) in EtOH (10 ml) gave 3,3′-(2,2′-(methylazanediyl)bis(ethane-2,1-diyl)bis(oxy))bis(N′-hydroxypropanimidamide) (1.28 g, 100%) as an oil.

Reaction of glycol derivative 3,3′-(ethane-1,2-diylbis(oxy))dipropanenitrile to give 3,3′-(ethane-1,2-diylbis(oxy))bis(N′-hydroxypropanimidamide):

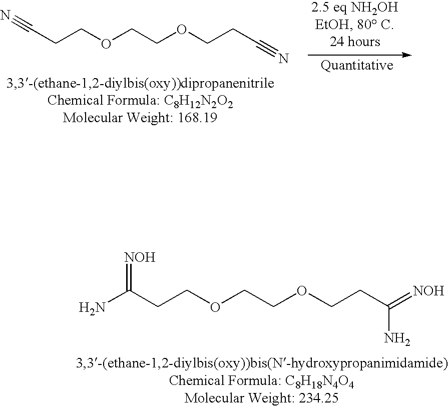

A solution of 3,3′-(ethane-1,2-diylbis(oxy))dipropanenitrile (1 g, 5 mmol) and NH₂OH (50% in water, 0.77 cm³, 12.5 mmol) in EtOH (10 cm³) was stirred at 80° C. for 24 hours and then at room temperature for 24 hours. The solvent and excess NH2OH were evaporated off and the residue was freeze-dried to give 3,3′-(ethane-1,2-diylbis(oxy))bis(N′-hydroxypropanimidamide) (1.33 g, 100%) as a viscous oil.

Reaction of 3,3′-(piperazine-1,4-diyl)dipropanenitrile to give 3,3′-(piperazine-1,4-diyl)bis(N′-hydroxypropanimidamide):

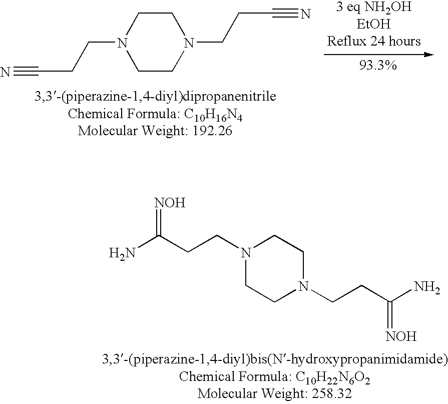

A solution of 3,3′-(piperazine-1,4-diyl)dipropanenitrile (1 g, 5.2 mmol) and NH₂OH (50% in water, 0.96 cm³, 15.6 mmol) in EtOH (10 cm³) were heated to reflux for 24 hours, after which the mixture was allowed to cool to room temperature. The solid formed was collected by filtration and dried in high vacuum line to give 3,3′-(piperazine-1,4-diyl)bis(N′-hydroxypropanimidamide) (1.25 g, 93.3%) as a white solid, deep 238° C. (brown colouration at >220° C.).

Reaction of cyanoethylated sorbitol compound with hydroxylamine to give 1,2,3,4,5,6-hexakis-O-[3-(hydroxyamino)-3-iminopropyl hexitol:

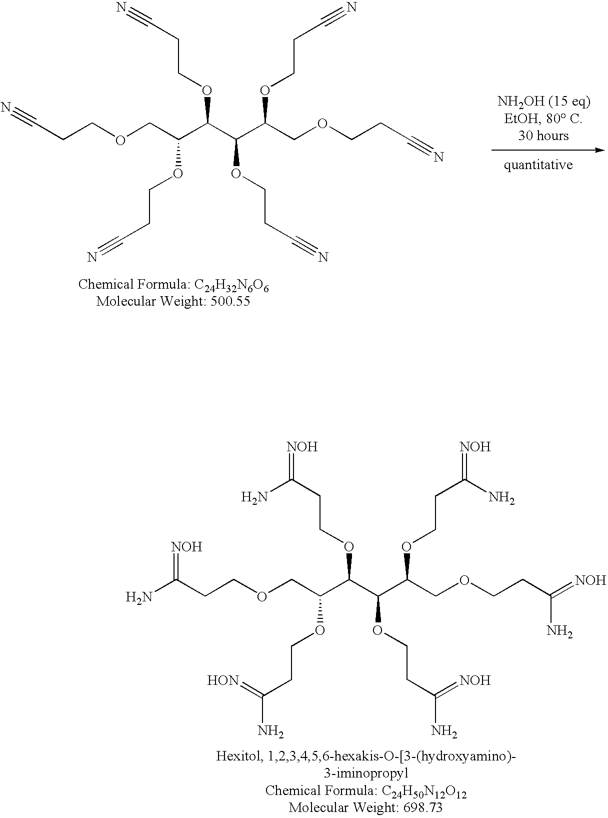

A solution of cyanoethylated product of sorbitol (0.48 g, 0.96 mmol) and NH₂OH (50% in water, 0.41 ml, 0.44 g, 6.71 mmol) in EtOH (5 ml) was stirred at 80° C. for 24 hours. Evaporation of solvent and NMR analysis of the residue showed incomplete conversion. The product was dissolved in water (10 ml) and EtOH (100 ml) and NH₂OH (0.5 g, 7.6 mmol) was added. The mixture was stirred at 80° C. for a further 7 hours. Removal of all volatiles after the reaction gave 1,2,3,4,5,6-hexakis-O-[3-(hydroxyamino)-3-iminopropyl hexitol, (0.67 g, 100%) as a white solid, mp 92-94° C. (decomposed).

Reaction of Benzonitrile to give N′-hydroxybenzimidamide:

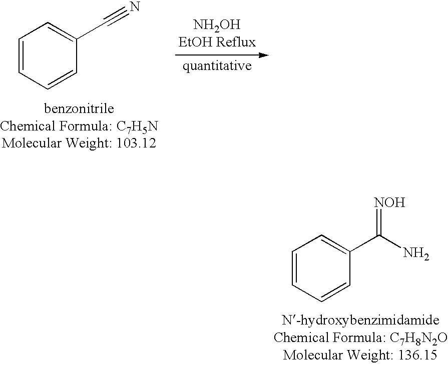

Benzonitrile (0.99 cm³, 1 g, 9.7 mmol) and hydroxylamine (50% in water, 0.89 cm³, 0.96 g, 14.55 mmol, 1.5 eq) were stirred under reflux in EtOH (10 cm³) for 48 hours. The solvent was evaporated under reduced pressure and water (10 cm³) was added to the residue. The mixture was extracted with dichloromethane (100 cm³) and the organic extract was evaporated under reduced pressure. The residue was purified by column chromatography to give the product N′-hydroxybenzimidamide (1.32 g, 100%) as a white crystalline solid, mp 79-81° C. (lit 79-80° C. This procedure is suitable for all starting materials bearing a benzene ring.

Reaction of 3-phenylpropionitrile to give N′-hydroxy-3-phenylpropanimidamide:

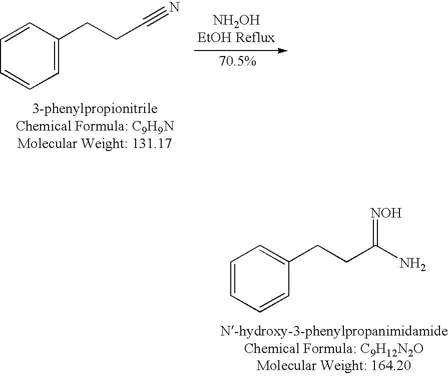

Phenylpropionitrile (1 g 7.6 mmol) was reacted with hydroxylamine (50% in water, 0.94 cm³, 15.2 mmol, 2 eq) in EtOH (7.6 cm³) in the same manner as in the preparation of N′-hydroxybenzimidamide (EtOAc used in extraction) to give the product N′-hydroxy-3-phenylpropanimidamide (0.88 g, 70.5%) as a white solid, mp 42-43° C.

Reaction of m-tolunitrile to give N′-hydroxy-3-methylbenzimidamide:

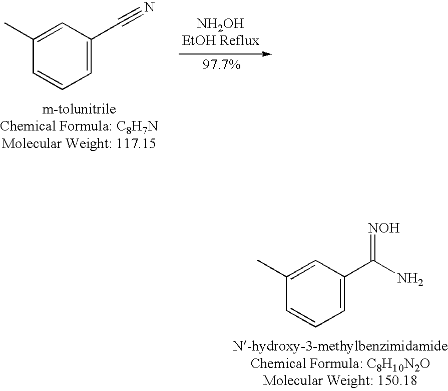

The reaction of m-tolunitrile (1 g, 8.54 mmol) and hydroxylamine (0.78 cm³, 12.8 mmol, 1.5 eq) in EtOH (8.5 cm³) was performed in the same manner as in the preparation of N′-hydroxybenzimidamide, to give the product N′-hydroxy-3-methylbenzimidamide (1.25 g, 97.7%) as a white solid, mp 92° C. (lit 88-90° C.).

Reaction of benzyl cyanide to give N′-hydroxy-2-phenylacetimidamide:

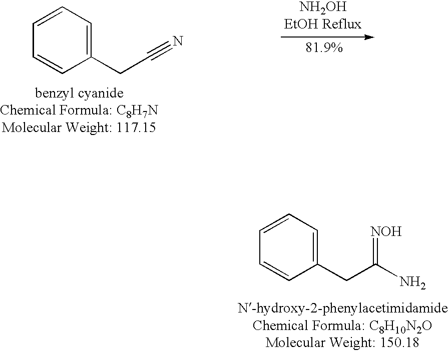

Benzyl cyanide (1 g, 8.5 mmol) and hydroxylamine (50% in water, 1.04 cm³, 17 mmol, 2 eq) in EtOH (8.5 cm³) were reacted in the same manner as in the preparation of N′-hydroxybenzimidamide (EtOAc used in extraction) to give the product N′-hydroxy-2-phenylacetimidamide (1.04 g, 81.9%) as a pale yellow solid, mp 63.5-64.5° C. (lit 57-59° C.).

Reaction of anthranilonitrile to give 2-amino-N′-hydroxybenzimidamide:

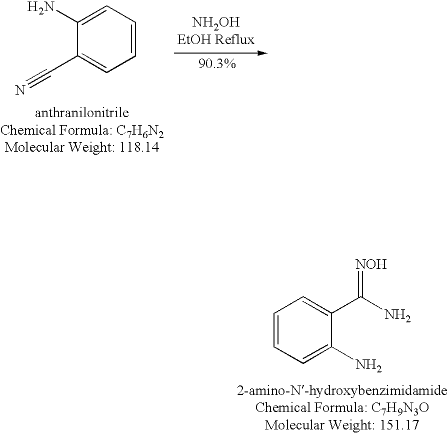

Anthranilonitrile (1 g, 8.5 mmol) and hydroxylamine (50% in water, 0.57 cm³, 9.3 mmol, 1.1 eq) in EtOH (42.5 cm³) were stirred under reflux for 24 hours, after which the volatiles were removed under reduced pressure and residue was partitioned between water (5 cm³) and CH₂Cl₂ (100 cm³). The organic phase was evaporated to dryness in the rotary evaporator followed by high vacuum line to give the product 2-amino-N′-hydroxybenzimidamide (1.16 g, 90.3%) as a solid, mp 85-86° C.

Reaction of phthalonitrile to give isoindoline-1,3-dione dioxime:

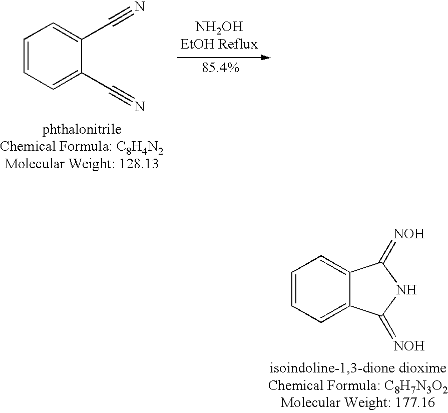

Phthalonitrile (1 g, 7.8 mmol) and hydroxylamine (1.9 cm³, 31.2 mmol, 4 eq) in EtOH (25 cm³) were stirred under reflux for 60 hours, after which the volatiles were removed under reduced pressure and the residue was washed with EtOH (2 cm³) and CH₂Cl₂ (2 cm³) to give the cyclised product isoindoline-1,3-dione dioxime (1.18 g, 85.4%) as a pale yellow solid, mp 272-275° C. (decomposed) (lit 271° C.).

Reaction of 2-cyanophenylacetonitrile to give the cyclised product 3-aminoisoquinolin-1(4H)-one oxime or 3-(hydroxyamino)-3,4-dihydroisoquinolin-1-amine:

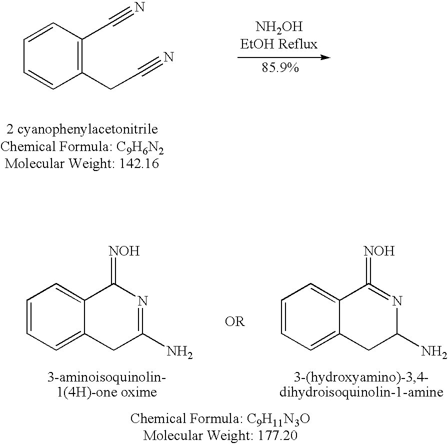

A solution of 2-cyanophenylacetonitrile (1 g, 7 mmol) and hydroxylamine (1.7 cm³, 28.1 mmol, 4 eq) in EtOH (25 cm³) were stirred under reflux for 60 hours, after which the volatiles were removed under reduced pressure. The residue was recrystallised from EtOH-water (1:4, 15 cm³) to give the cyclised product 3-aminoisoquinolin-1(4H)-one oxime or 3-(hydroxyamino)-3,4-dihydroisoquinolin-1-amine (1.15 g, 85.9%) as a solid, mp 92.5-94.5° C.

Reaction of cinnamonitrile to give N′-hydroxycinnamimidamide:

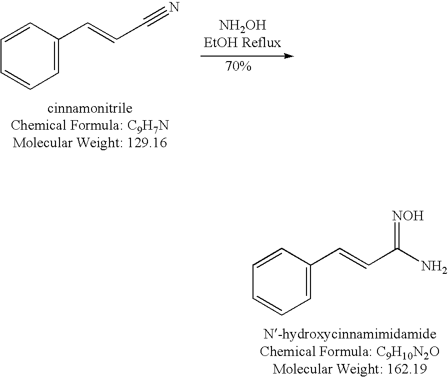

Cyanoethylation of piperizine:

Cinnamonitrile (1 g, 7.74 mmol) and hydroxylamine (0.71 cm³, 11.6 mmol, 1.5 eq) were reacted in EtOH (7 cm³) as described for AO6 (two chromatographic separations were needed in purification) to give N′-hydroxycinnamimidamide (0.88 g, 70%) as a light orange solid, mp 85-87° C. (lit 93° C.).

Reaction of 5-cyanophthalide to give the product N′-hydroxy-1-oxo-1,3-dihydroisobenzofuran-5-carboximidamide:

Cyanoethylation of piperizine:

A solution of 5-cyanophthalide (1 g, 6.28 mmol) and hydroxylamine (50% in water, 0.77 cm³, 0.83 g, 12.6 mmol, 2 eq) in EtOH (50 cm³) was stirred at room temperature for 60 hours and then under reflux for 3 hours. After cooling to room temperature and standing overnight, the solid formed was collected by filtration and dried in high vacuum line to give the product N′-hydroxy-1-oxo-1,3-dihydroisobenzofuran-5-carboximidamide (1.04 g, 86.2%) as a white solid, mp 223-226° C. (decomposed).

Reaction of 4-chlorobenzonitrile to give the product 4-chloro-N′-hydroxybenzimidamide:

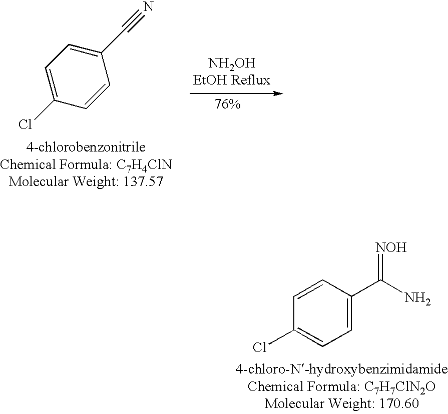

A solution of 4-chlorobenzonitrile (1 g, 7.23 mmol) and hydroxylamine (50% in water, 0.67 cm³, 10.9 mmol, 1.5 eq) in EtOH (12.5 cm³) was stirred under reflux for 48 hours. The solvent was removed under reduced pressure and the residue was washed with CH₂Cl₂ (10 cm³) to give the product 4-chloro-N′-hydroxybenzimidamide (0.94 g, 76%) as a white solid, mp 133-135° C.

Reaction of 3-(phenylamino)propanenitrile to give N′-hydroxy-3-(phenylamino)propanimidamide:

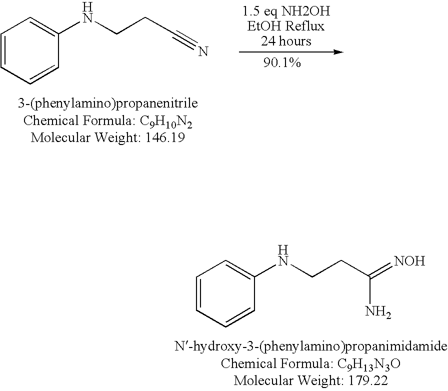

A solution of 3-(phenylamino)propanenitrile (1 g, 6.84 mmol) and NH₂OH (50% in water, 0.63 cm³, 10.26 mmol) in EtOH (10 cm³) were heated to reflux for 24 hours, after which the solvent and excess hydroxylamine were removed by rotary evaporator. To the residue was added water (10 cm³) and the mixture was extracted with CH₂Cl₂ (100 cm³). The extracts were concentrated under reduced pressure and the residue was purified by column chromatography (silica, Et₂O) to give N′-hydroxy-3-(phenylamino)propanimidamide (0.77 g, 62.8%) as a white solid, mp 93-95° C. (lit mp 91-91.5° C.).

Reaction of 4-pyridinecarbonitrile to give the product N′-hydroxyisonicotinimidamide:

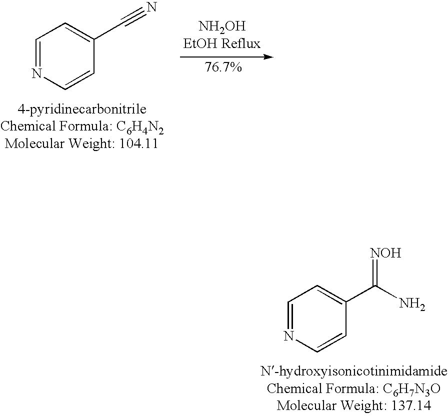

Pyridinecarbonitrile (1 g, 9.6 mmol) and hydroxylamine (50% in water, 0.88 cm³, 14.4 mmol, 1.5 eq) in EtOH (10 cm³) were stirred under reflux for 18 hours, after which the volatiles were removed under reduced pressure and the residue was recrystallised from EtOH to give the product N′-hydroxyisonicotinimidamide (1.01 g, 76.7%) as a solid, mp 203-205° C.

Compounds from cyanoethylation of isobutyraldehyde, diethylmalonate, cyanoacetamide, glycine anhydride, glycine and malononitrile and subsequent reacting with hydroxylamine do not produce the corresponding amidoximes. However, these mono and multi-cyanoethylated products show to have good chelating property on their own and can be used in cleaning residue from copper surface.

The following structure depicts metal complexing using amidoxime compounds.

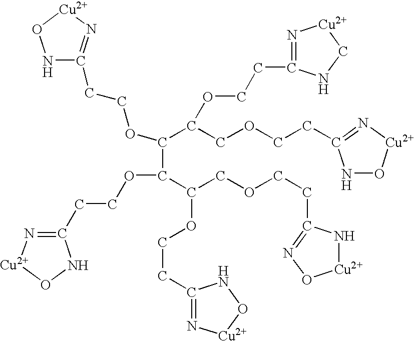

Amidoxime chelating agents are suitable substitutes in many cases for organic carboxylic acids, organic carboxylic ammonium salt or an amine carboxylates being used in cleaning formulations and processes.

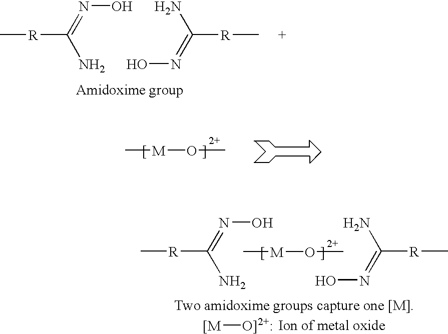

With reference to the present invention, as hereinafter more fully described, the claimed compounds can be applied to applications in the state of the art forming a background to the present invention includes the following U.S. patents, the disclosures of which hereby are incorporated herein, in their respective entireties.

### EXAMPLES OF EMBODIMENTS OF THE PRESENT INVENTION

Five samples of hydroxylamine free base (50%) solution were contacted with 1 ppm, 5 ppm, 10 ppm, 25 ppm and 50 ppm of FeCl₃. The solutions were then immersed in a constant temperature water bath which was maintained at 50° C. Samples were taken out after 24 hours and 48 hours for remaining hydroxylamine contents.

Decomposition Experiment of Hydroxylamine with Iron III Chloride

| | Initial Test | | 24 Hour test @ 50° C. | | 48 Hour test @ 50° C. | |
|---|---|---|---|---|---|---|
| Samples Spike | | Average | | Average | | Average |
| with Fe(III) | Average | Hydroxylamine | Average | Hydroxylamine | Average | Hydroxylamine |
| Chloride | Fe(ppb) | (wt %) | Fe(ppb) | (wt %) | Fe(ppb) | (wt %) |
| 0 ppm | 9 | 49.924 | 9 | 50.362 | 9 | 50.363 |
| 1 ppm | 1321 | 49.507 | 1246 | 50.051 | 1372 | 50.229 |
| 5 ppm | 5341 | 49.199 | 5458 | 50.472 | 4723 | 49.615 |
| 10 ppm | 8739 | 49.114 | 10485 | 49.308 | 9652 | 48.927 |
| 25 ppm | 25230 | 48.519 | 23935 | 47.747 | 23970 | 47.143 |
| 50 ppm | 49350 | 48.475 | 45015 | 33.470 | 40627 | 28.307 |

The results showed that at 50° C. hydroxylamine contaminated with 50 ppm of Fe(III) chloride decomposed by 57% in 48 hours.

### Example 2

### Comparison Example to U.S. Pat. No. 3,480,391.

50 ppm of FeCl₃ solution was added to the following hydroxylamine solution stabilized with various nitriles, amidoxime and hydroxamic acid compounds. The solutions were placed in a 50° C. water bath for 24 hours. Hydroxylamine concentration was analyzed after 24 hours using titration method.

| | | | | Physical | |
|---|---|---|---|---|---|
| ID | Group | Compound | MW | State | Solution |
| AO3 | Nitrile | 3-Hydroxypropionitrile | 71.04 | Liquid | 5% |
| AO8 | Nitrile | 3,3′ iminodipropionitrile | 123.16 | Liquid | 5% |
| AO7 | Reaction product from | 3,3′,3″,3′″-(ethane-1,2- | 404.5 | Solid | 1% |
| | cyanoethylation of | diylbis(azanetriyl))tetrakis(N′- | | | |
| | ethylenediamine followed | hydroxypropanimidamide) | | | |
| | by its conversion to | | | | |
| | amidoxime | | | | |

| | | | | |
|---|---|---|---|---|
| Hydroxamic acid | (Z)-4-hydroxyamino)-4-oxobut- | 131.09 | Solid | 1% |
| (U.S. Pat. No. 3,480,392) | 2-enoic acid | | | |
| Nitrile (U.S. Pat. No. 3,480,391) | Benzonitrile | 103.12 | Solid | 1% |

### Results:

| | | | | HDA(R) | Measured | |
|---|---|---|---|---|---|---|
| | Weight | | HDA(R) | Remained | (after 24 | |
| Test Result | added | Moles | Consumed | (Before) | Hours) | Change |
| AO3 | 5 | 0.070 | 2.325 | 47.675 | 45.632 | 4.3% |
| AO8 | 5 | 0.041 | 2.682 | 47.318 | 44.812 | 5.3% |
| AO7 | 1 | 0.012 | 1.225 | 48.775 | 48.655 | 0.2% |
| Hydroxamic acid | 1 | 0.000 | 0.000 | 50.000 | 47.854 | 4.3% |
| (U.S. Pat. No. 3,480,392) | | | | | | |
| Nitrile | 1 | 0.010 | 0.320 | 49,680 | 46.909 | 5.6% |
| (U.S. Pat. No. 3,480,391) | | | | | | |

All of the tested compounds provided further stability of the hydroxylamine. It was evident by comparing Example 1 results with the Example 2 results which hydroxylamine samples degraded about 30%. Nitriles with a molecular weight of less than about 200 were observed to degrade the hydroxylamine more than compound AO7, which was prepared from the reaction of ethylenediamine with acrylonitrile followed by conversion to an amidoxime with chemical name of 3,3′,3″,3′″-(ethane-1,2-diylbis(azanetriyl))tetrakis(N′-hydroxypropanimidamide).

### Example 3

| | |
|---|---|
| AmiSorb ™ DS6 | 60% solution of 1,2,3,4,5,6-hexakis-O-[3- |
| | (hydroxyamino)-3-iminopropyl Hexitol solution |
| DGA | Diglycolamine |
| MEA | Monoethanolamine |
| DMSO | Dimethylsulfoxide |
| TMAH | 25% Tetramethylammonium Hydroxide |
| | solution |
| Choline | 40% Choline Hydroxide solution |

| | | Hydroxylamine | | | | | | | | |
|---|---|---|---|---|---|---|---|---|---|---|
| | AmiSorb ™ | Free base | | | | | | Hydroxylamine | | |
| # | DS6 | (50%) | DGA | MEA | DMSO | TMAH | Choline | Before | After | Change |
| A | | 40 | 60 | | | | | 20 | 19.4 | 3.0% |
| B | 5 | 40 | 55 | | | | | 20 | 20 | 0.0% |
| C | | 40 | | 60 | | | | 20 | 16.5 | 17.5% |
| D | 5 | 40 | | 55 | | | | 20 | 20 | 0.0% |
| E | | 5 | | | 70 | 25 | | 2.5 | 2.3 | 4.6% |
| F | 5 | 5 | | | 65 | 25 | | 2.5 | 2.4 | 2.9% |
| G | | 5 | | | 70 | | 25 | 2.5 | 2.5 | 0.0% |
| H | 5 | 5 | | | 65 | | 25 | 2.5 | 2.5 | 0.0% |

Solution containing amidoxime molecule of 1,2,3,4,5,6-hexakis-O-[3-(hydroxyamino)-3-iminopropyl hexitol solution provided better stability to the cleaning solutions than those without any stabilizers.

### Example 4

The following nitrile compounds with various carbons and molecular weights are introduced to hydroxylamine freebase solution. From each of the samples we extracted 10 ml and added 100 μl of a Fe stock (1000 ppm), an effective dose of 10 ppm of Fe. After 24 hours at 50° C., samples were analyzed for HDA %. The results show that nitrile compounds react with hydroxylamine to form the corresponding amidoxime molecules and further stabilize the hydroxylamine solution even with the introduction of 10 ppm of iron to the solution.

| | | | Chemical | Molecular |
|---|---|---|---|---|
| ID | Nucleophile | Nitriles | Formula | Weight |
| U.S. Pat. No. 3,48,0931 | | benzonitrile | C7H5N | 103.12 |
| CE1 | Sorbitol | 1,2,3,4,5,6-hexakis-O-(2-cyanoetyl)hexitol | C24H32N6O6 | 500.55 |
| CE7 | ethylenediamine | 3,3′,3″,3′″-(ethane-1,2- | C14H20N6 | 272.35 |
| | | diylbis(azanetriyl))tetrapropanenitrile | | |
| CE28 | ethylene glycol | 3,3′-(ethane-1,2-diylbis(oxy))dipropanenitrile | C8H12N2O2 | 168.19 |
| CE41 | ammonia | 3,3′,3″-nitrilotripropanenitrile | C9H12N4 | 176.22 |
| CE43 | glycine | 2-(2-cyanoethylamino)acetic acid | C5H8N2O2 | 128.13 |
| CE44 | glycine | 2-(bis(2-cyanoethyl)amino)acetic acid | C8H11N3O2 | 181.19 |
| CE45 | malononitrile | 1,3,3,5-tetracarbonitrile | C9H8N4 | 172.19 |
| CE46 | cyanoacetamide | 2,4-dicyano-2-(2-cyanoethyl)butanamide | C9H10N4O | 190.2 |
| CE47 | Pentaerythritol | 3,3′-(2,2-bis((2-cyanoethoxy)methyl)propane-1,3- | C17H24N4O4 | 348.4 |
| | | diyl)bis(oxy)dipropanenitrile | | |
| CE48 | N-methyldiethanolamine | 3,3′-(2,2′-(methylazanediyl)bis(ethane-2,1- | C11H19N3O2 | 225.29 |
| | | diyl)bis(oxy))dipropanenitrile | | |
| CE49 | glycine anhydride | 3,3′-(2,5-dioxopiperazine-1,4- | C10H12N4O2 | 220.23 |
| | | diyl)dipropanenitrile | | |
| CE50 | acetamide | N,N-bis(2-cyanoethyl)acetamide | C8H11N3O | 165.19 |
| CE51 | anthranilonitrile | 3,3′-(2-cyanophenylazanediyl)dipropanenitrile | C13H12N4 | 224.26 |
| CE52 | diethanolamine | 3,3′-(2,2′-(2-cyanoethylazanediyl)bis(ethane-2,1- | C13H20N4O2 | 264.32 |
| | | diyl)bis(oxy))dipropanenitrile | | |

| | | | Theoretical | | | | | |
|---|---|---|---|---|---|---|---|---|
| | | | | NH₂OH | | Measured | | |
| | # of CN | | moles of | consumed | NH₂OH | | After 24 | % |
| Compound | groups | MW | CE/g soln | (%) | (%) | 0 hour | hours | Change |
| CE1 | 6 | 500.55 | 0.000020 | 0.40 | 49.10 | 48.22 | 47.47 | 2% |
| CE7 | 4 | 272.36 | 0.000037 | 0.49 | 49.01 | 48.95 | 48.82 | 0% |
| CE28 | 2 | 168.20 | 0.000059 | 0.39 | 49.11 | 48.99 | 48.71 | 1% |
| CE41 | 3 | 176.23 | 0.000057 | 0.56 | 48.94 | 47.96 | 47.81 | 0% |
| CE43 | 1 | 128.13 | 0.000078 | 0.26 | 49.24 | 48.29 | 48.35 | 0% |
| CE44 | 2 | 181.20 | 0.000055 | 0.36 | 49.14 | 47.67 | 46.3 | 3% |
| CE45 | 4 | 172.19 | 0.000058 | 0.77 | 48.73 | 48.32 | 47.48 | 2% |
| CE46 | 3 | 190.93 | 0.000052 | 0.52 | 48.98 | 48.1 | 44.95 | 7% |
| CE47 | 4 | 348.41 | 0.000029 | 0.38 | 49.12 | 48 | 47.75 | 1% |
| CE48 | 2 | 225.30 | 0.000044 | 0.29 | 49.21 | 47.8 | 48.1 | 0% |
| CE49 | 2 | 220.23 | 0.000045 | 0.30 | 49.20 | 49.43 | 49.74 | 0% |
| CE50 | 2 | 165.20 | 0.000061 | 0.40 | 49.10 | 46.92 | 46.17 | 2% |
| CE51 | 3 | 224.27 | 0.000045 | 0.44 | 49.06 | 48.49 | 48.6 | 0% |
| CE52 | 3 | 264.33 | 0.000038 | 0.37 | 49.13 | 48.71 | 49.66 | 0% |

List of specific amidoxime compounds prepared from nitrites:

| Nitrile | Amidoxime |
|---|---|
| 3- | N′,3-dihydroxypropanimidamide |
| hydroxypropionitrile | |
| Acetonitrile | NN′-hydroxyacetimidamide |
| 3-methyl- | N′-hydroxy-3-(methylamino)propanimidamide |
| aminopropionitrile | |
| Benzonitrile | N′-hydroxybenzimidamide |
| 3,3′ imino- | 3,3′-azanediylbis(N′-hydroxypropanimidamide) |
| dipropionitrile | |
| octanonitrile | N′-hydroxyoctanimidamide |
| 3- | N′-hydroxy-3-phenylpropanimidamide |
| phenylpropionitrile | |
| ethyl 2-cyanoacetate | 3-amino-N-hydroxy-3-(hydroxyimino)propanamide |
| 2-cyanoacetic acid | 3-amino-3-(hydroxyimino)propanoic acid |
| 2-cyanoacetamide | 3-amino-3-(hydroxyimino)propanamide |
| adiponitrile | N′1,N′6-dihydroxyadipimidamide |
| sebaconitrile | N′1,N′10-dihydroxydecanebis(imidamide) |
| 4- | N′-hydroxyisonicotinimidamide |
| pyridinecarbonitrile | |
| m-tolunitrile | N′-hydroxy-3-methylbenzimidamide |
| phthalonitrile | isoindoline-1,3-dione dioxime |
| glycolonitrile | N′,2-dihydroxyacetimidamide |
| chloroacetonitrile | 2-chloro-N′-hydroxyacetimidamide |
| benzyl cyanide | product N′-hydroxy-2-phenylacetimidamide |
| Anthranilonitrile | 2-amino-N′-hydroxybenzimidamide |
| 3,3′ imino- | 2,2′-azanediylbis(N′-hydroxyacetimidamide) |
| diacetonitrile | |
| 5-cyanophthalide | N′-hydroxy-1-oxo-1,3-dihydroisobenzofuran-5- |
| | carboximidamide |
| 2-cyanophenyl- | 3-aminoisoquinolin-1(4H)-one oxime or 3- |
| acetonitrile | (hydroxyamino)-3,4-dihydroisoquinolin-1-amine |
| cinnamonitrile | N′-hydroxycinnamimidamide |
| 5-hexynenitrile | 4-cyano-N′-hydroxybutanimidamide |
| 4-chlorobenzonitrile | 4-chloro-N′-hydroxybenzimidamide |

List of specific amidoxime compounds prepared from nitriles by cyanoethylation of nucleophilic compounds:

| Nucleophilic | Cyanoethylated | Amidoxime from cyanoethylated |
|---|---|---|
| compounds | Compounds | compounds |
| Sorbitol | | 1,2,3,4,5,6-hexakis-O-[3- |
| | | (hydroxyamino)-3-iminopropyl |
| | | Hexitol, |
| ethylenediamine | 3,3′,3″,3′″-(ethane-1,2- | 3,3′,3″,3′″-(ethane-1,2- |
| | diylbis(azanetriyl))tetra- | diylbis(azanetriyl))tetrakis(N′- |
| | propanenitrile | hydroxypropanimidamide) |
| ethylene glycol | 3,3′-(ethane-1,2- | 3,3′-(ethane-1,2-diylbis(oxy))bis(N′- |
| | diylbis(oxy)) | hydroxypropanimidamide) |
| | dipropanenitrile | |
| diethylamine | 3-(diethylamino) | 3-(diethylamino)-N′- |
| | propanenitrile | hydroxypropanimidamide |
| piperazine | 3,3′-(piperazine-1,4-diyl) | 3,3′-(piperazine-1,4-diyl)bis(N′- |
| | dipropanenitrile | hydroxypropanimidamide) |
| 2-ethoxy | 3-(2-ethoxyethoxy) | 3-(2-ethoxyethoxy)-N′- |
| ethanol | propanenitrile | hydroxypropanimidamide |
| 2-(2-dimethyl | 3-(2-(2-(dimethylamino) | 3-(2-(2- |
| aminoethoxy) | ethoxy) ethoxy) | (dimethylamino)ethoxy)ethoxy)-N′- |
| ethanol | propanenitrile | hydroxypropanimidamide |
| aniline | 3-(phenylamino) | N′-hydroxy-3- |
| | propanenitrile | (phenylamino)propanimidamide |
| ammonia | 3,3′,3″- | 3,3′,3″-nitrilotris(N′- |
| | nitrilotripropanenitrile | hydroxypropanimidamide) |
| Pentaerythritol | 3,3′-(2,2-bis((2- | 3,3′-(2,2-bis((3-(hydroxyamino)-3- |
| | cyanoethoxy) | iminopropoxy)methyl)propane-1,3- |
| | methyl)propane-1,3-diyl) | diyl)bis(oxy)bis(N- |
| | bis(oxy)dipropanenitrile | hydroxypropanimidamide) |
| N-methyl | 3,3′-(2,2′- | 3,3′-(2,2′- |
| diethanolamine | (methylazanediyl) | (methylazanediyl)bis(ethane-2,1- |
| | bis(ethane-2,1-diyl) | diyl)bis(oxy))bis(N′- |
| | bis(oxy))dipropanenitrile | hydroxypropanimidamide) |
| acetamide | N,N-bis(2- | N,N-bis(3-amino-3- |
| | cyanoethyl)acetamide | (hydroxyimino)propyl)acetamide |
| anthranilonitrile | 3,3′-(2- | 3,3′-(2-(N′- |
| | cyanophenylazanediyl) | hydroxycarbamimidoyl)phenylazane- |
| | dipropanenitrile | diyl)bis(N′-hydroxypropanimidamide) |
| diethanolamine | 3,3′-(2,2′-(2- | 3,3′-(2,2′-(3-amino-3- |
| | cyanoethylazanediyl) | (hydroxyimino)propylazanediyl)bis |
| | bis(ethane-2,1-diyl) | (ethane-2,1-diyl))bis(oxy)bis(N′- |
| | bis(oxy))dipropanenitrile | hydroxypropanimidamide) |

While the invention has been described and illustrated herein by references to various specific materials, procedures and examples, it is understood that the invention is not restricted to the particular combinations of materials and procedures selected for that purpose. Numerous variations of such details can be implied as will be appreciated by those skilled in the art. It is intended that the specification and examples be considered as exemplary, only, with the true scope and spirit of the invention being indicated by the following claims. All references, patents, patent applications and other publications referred to in this application are herein incorporated by reference in their entireties.

1. A method of preventing degradation of or stabilizing hydroxylamine, comprising contacting the hydroxylamine with an effective amount of an amidoxime compound, wherein the amidoxime compound is prepared from a reaction of hydroxylamine and a nitrile compound.

2. The method of claim 1, wherein the hydroxylamine is present as an aqueous solution.

3. The method of claim 1, wherein the amidoxime has any one of the following structures: 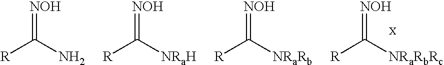
or tautomers thereof wherein X is a counterion and R, Rₐ, R_{b} and R_{c} are independently selected from alkyl, heteroalkyl, aryl and heteroaryl, wherein the alkyl, heteroalkyl, aryl and heteroaryl are optionally substituted.

4. The method of claim 3, wherein R is an optionally substituted alkyl group.

5. The method of claim 3, wherein R is an optionally substituted heteroalkyl group.

6. The method of claim 3, wherein R contains more than 10 carbons.

7. The method of claim 2, wherein each of the structures has a molecular weight of above 200.

8. The method of claim 1, wherein the amidoxime has the following structure: 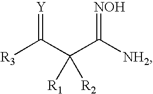
wherein R₁ and R₂ are independently selected from hydrogen, alkyl, heteroalkyl, aryl and heteroaryl; R₃ is alkyl, heteroalkyl, aryl and heteroaryl, wherein the alkyl, heteroalkyl, aryl and heteroaryl are optionally substituted; and Y is O, NH or NOH.

9. The method of claim 1, wherein the amidoxime has the following structure: 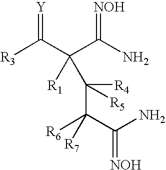
wherein R₁, R₂, R₄, R₅, R₆ and R₇ are independently selected from hydrogen, alkyl, heteroalkyl, aryl and heteroaryl; R₃ is alkyl, heteroalkyl, aryl and heteroaryl, wherein the alkyl, heteroalkyl, aryl and heteroaryl are optionally substituted; and Y is O, NH or NOH.

10. The method of claim 1 wherein the amidoxime is selected from the group consisting of
1,2,3,4,5,6-hexakis-O-[3-(hydroxyamino)-3-iminopropyl hexitol;
3,3′,3″,3′″-(ethane-1,2-diylbis(azanetriyl))tetrakis(N′-hydroxypropanimidamide);
3,3′-(ethane-1,2-diylbis(oxy))bis(N′-hydroxypropanimidamide);
3-(diethylamino)-N′-hydroxypropanimidamide;
3,3′-(piperazine-1,4-diyl)bis(N′-hydroxypropanimidamide);
3-(2-ethoxyethoxy)-N′-hydroxypropanimidamide;
3-(2-(2-(dimethylamino)ethoxy)ethoxy)-N′-hydroxypropanimidamide;
N′-hydroxy-3-(phenylamino)propanimidamide;
3,3′,3″-nitrilotris(N′-hydroxypropanimidamide);
3,3′-(2,2-bis((3-(hydroxyamino)-3-iminopropoxy)methyl)propane-1,3-diyl)bis(oxy)bis(N-hydroxypropanimidamide);
3,3′-(2,2′-(methylazanediyl)bis(ethane-2,1-diyl)bis(oxy))bis(N′-hydroxypropanimidamide);
N,N-bis(3-amino-3-(hydroxyimino)propyl)acetamide;
3,3′-(2-(N′-hydroxycarbamimidoyl)phenylazanediyl)bis(N′-hydroxypropanimidamide);
3,3′-(2,2′-(3-amino-3-(hydroxyimino)propylazanediyl)bis(ethane-2,1-diyl))bis(oxy)bis(N′-hydroxypropanimidamide);
N′,3-dihydroxypropanimidamide;
NN′-hydroxyacetimidamide;
N′-hydroxy-3-(methylamino)propanimidamide;
3,3′-azanediylbis(N′-hydroxypropanimidamide);
3-amino-3-(hydroxyimino)propanoic acid;
3-amino-3-(hydroxyimino)propanamide;
N′1,N′10-dihydroxydecanebis(imidamide);
N′-hydroxyisonicotinimidamide;
2-dihydroxyacetimidamide;
2-chloro-N′-hydroxyacetimidamide;
2-amino-N′-hydroxybenzimidamide;
2,2′-azanediylbis(N′-hydroxyacetimidamide);
N′-hydroxy-1-oxo-1,3-dihydroisobenzofuran-5-carboximidamide;
3-aminoisoquinolin-1(4H)-one oxime;
3-(hydroxyamino)-3,4-dihydroisoquinolin-1-amine;
N′-hydroxycinnamimidamide;
4-chloro-N′-hydroxybenzimidamide;
and salts thereof.

11. The method of claim 1, wherein the nitrile compound is prepared from cyanoethylation of nucleophilic compounds with acrylonitrile.

12. The method of claim 11, wherein the nucleophilic compounds are selected from the group consisting of
a. compounds containing one or more —OH or —SH groups;
b. compounds containing one or more —NH— groups;
c. ketones or aldehydes possessing a —CH—, —CH₂—, or —CH₃ group adjacent to the carbonyl group; and
d. malonic esters, malonamide and cyanoacetamide.

13. The method of claim 12, wherein the compounds containing one or more —OH or —SH groups are alcohols, phenols, oximes, hydrogen sulphide and thiols.

14. The method of claim 12, wherein the compounds containing one or more —NH— groups are ammonia, primary and secondary amines, hydrazines, and amides.
